(19) United States
(12) Patent Application Publication
(10) Pub. No.: US 2005/0130306 A1
(43) Pub. Date: Jun. 16, 2005

(54) Viral vectors whose replication and, optionally, passenger gene are controlled by a gene switch activated by heat in the presence or absence of a small molecule regulator

(76) Inventor: Richard Voellmy, Pully (CH)

Correspondence Address: Richard Voellmy, Avenue des Cerisiers 39B, Pully, 1009, CH

(21) Appl. No.: 10/731,961

(22) Filed: Dec. 10, 2003

(51) Int. Cl. C12N 15/861; C12N 7/00

(52) U.S. Cl. 435/456; 435/235.1

(57) ABSTRACT

The present invention relates to conditionally replicating viruses or pairs of viruses containing a gene switch that is activatable by transient heat or other proteotoxic stress in the presence or absence of a small molecule regulator. The gene switch controls the expression of a gene for a protein required for efficient viral replication and may also control the activity of a passenger gene.

### TECHNICAL FIELD

The present invention relates to viral vectors whose replication and, optionally, passenger gene expression are regulated by a gene switch that is dually controlled by heat and a small molecule regulator.

### BACKGROUND OF THE INVENTION

There are numerous situations in biological research, ex vivo cell therapies and gene therapies of experimental animals and, eventually, humans, where careful control of the distribution and expression of an introduced gene or a replicating virus is critically important. Viral and non-viral vectors provide means for delivering genes to cells, tissues and organs. However, this delivery is, typically, not specific for any particular cell type, tissue or organ location. Previously, so-called tissue-specific promoters were used to restrict virus replication or expression of a gene delivered by a vector to cells of a specific type. A shortcoming of this approach is that is uncertain whether a “tissue-specific” promoter really exists. Although a promoter may have a much stronger activity in a particular cell type than in others, it is likely to display at least some activity in some other cells. Furthermore, the rate of transcription supported by a chosen “tissue-specific” promoter, which rate is an intrinsic property of the promoter, may limit its usefulness. Other approaches for controlling expression of therapeutic genes included the use of two-component gene switches that are activated or inactivated by a small molecule regulator. Well-known switches are controlled by tetracycline, ecdysterone, mifepristone or rapamycin. While these switches may allow for stringent on-off regulation of gene activity, basal gene activity cannot be controlled and intermediate levels of gene activity are typically difficult to achieve. Moreover, gene expression cannot be locally restricted by a small molecule regulator that readily diffuses through tissues. Because an activating heat treatment can be directed to a target tissue, heat shock gene promoters should provide an effective means for restricting expression of a gene delivered by a viral or other vector to a target tissue. These promoters have also been suggested to be useful for controlling viral replication (U.S. 09/850270). Some of these promoters, in particular a human hsp70B gene promoter isolated and characterized by the inventor, have minimal basal activity and a heat-induced activity that can be a thousand fold greater than basal activity. Such promoters should be particularly useful for achieving strict regulation of localized gene expression or virus replication. However, there is a downside to using promoters that are activated by transient heat and other proteotoxic stresses. After introduction into an experimental animal or a human, inadvertent activation of these promoters can occur during a fever or ischemia/reperfusion, exposure to oxidant or other proteotoxic agent, or, possibly, even hormonal stress. The confounding effects of such inadvertent, non-directed activation of virus replication or gene expression will be unacceptable in many applications. Gene switches that are only activated by a combination of heat (or other proteotoxic stress) and a small molecule regulator were previously described and were suggested to be useful to achieve spatial as well as temporal control of therapeutic gene expression (U.S. Pat. No. 6,342,596; U.S. patent application 10/046420).

### SUMMARY OF THE INVENTION

The present invention relates to a modified, conditionally replication-competent virus whose genome includes a gene switch activatable in an infected cell by exposure of the cell to heat and a small molecule regulator, the gene switch controlling the expression of a gene for a viral protein required for efficient replication of the modified virus. The invention similarly relates to a modified, conditionally replication-competent virus whose genome includes a gene switch that is activated in an infected cell by exposure of the cell to heat and is repressed by exposure of the cell to a small molecule regulator, the gene switch controlling the expression of a gene for a viral protein required for efficient replication of the modified virus. A modified, conditionally replication-competent virus of the invention may further include a passenger gene. Expression of the passenger gene can also be regulated by the gene switch that controls viral replication. Preferably, a modified, conditionally replication-competent virus is derived from a member of the Adenoviridae, Herpesviridae, and Retroviridae families. Most preferably, a modified, conditionally replication-competent virus is derived from a member of the Adenoviridae family. If the modified, conditionally replication-competent virus is an adenovirus, the genes that can be controlled by the gene switch include the E1A, E1B and E4 genes. The gene switch that controls the expression of one or more viral genes, whose product is required for or facilitates viral replication, comprises two components. The first component is a gene for a transactivator that is activated or inhibited by a small molecule regulator. In preferred embodiments, expression of the transactivator gene is controlled by a heat shock promoter activatable by transient heat or a transient proteotoxic stress through the intermediary of endogenous heat shock factor 1 (HSF1), or by a tandem or hybrid promoter activatable by transient heat or proteotoxic stress through the intermediary of endogenous HSF1 and by active transactivator, The second component of the switch is a promoter activated by the active form of the transactivator, which promoter is functionally linked to a viral or passenger gene to be regulated. In alternative embodiments, the first component is a transactivator gene that is expressed continuously (constitutively) in a host cell. The second component can be a modified heat shock promoter (including an appropriate RNA leader region) that is activated by transient heat or other proteotoxic stress and repressed by the transactivator. Binding of a small molecule regulator to the transactivator can, respectively, inhibit or enable its repressing function. Hence, the resulting gene switch is active in cells exposed to heat or proteotoxic stress in the presence or absence, respectively, of the small molecule regulator. The second component can also be a modified (or partial) heat shock promoter that requires co-activation by transactivator and endogenous HSF1. In this case, transactivator is activated by a bound small molecule regulator.

The invention also relates to a pair of modified viruses whose combined genomes contain all genetic information required for conditional replication of the virus pair; including a gene switch activatable in an infected cell by exposure of the cell to heat and a small molecule regulator, the gene switch controlling the expression of a gene for a viral protein required for efficient replication of the virus pair. The invention further concerns a pair of modified viruses whose combined genomes contain all genetic information required for conditional replication of the virus pair; including a gene switch that is activated in an infected cell by exposure of the cell to heat and is repressed by exposure of the cell to a small molecule regulator, the gene switch controlling the expression of a gene for a viral protein required for efficient replication of the virus pair. A pair of modified viruses of the invention may further include a passenger gene that is inserted into the genome of one or the other virus of the pair. The passenger gene can also be controlled by the gene switch that controls viral replication. Preferably, the modified viruses of a pair are modified members of the Adenoviridae, Herpesviridae, and Retroviridae families. Most preferably, each virus of a pair is derived from a member of the Adenoviridae family. If both viruses of a pair of the invention are modified adenoviruses, the genes that can be controlled by the gene switch include the E1A, E1B and E4 genes. The gene switch that controls the expression of one or more viral genes, whose product is required for or facilitates viral replication, comprises two components. The first component is a gene for a transactivator that is activated or inhibited by a small molecule regulator. In preferred embodiments, expression of the transactivator gene is controlled by a heat shock promoter activatable by transient heat or a transient proteotoxic stress through the intermediary of endogenous heat shock factor 1 (HSF1), or by a tandem or hybrid promoter activatable by transient heat or proteotoxic stress through the intermediary of endogenous HSF1 and by active transactivator, The second component of the switch is a promoter activated by the active form of the transactivator, which promoter is functionally linked to a viral or passenger gene to be regulated. In alternative embodiments, the first component is a transactivator gene that is expressed continuously (constitutively) in a host cell. The second component can be a modified heat shock promoter (including an appropriate RNA leader region) that is activated by transient heat or other proteotoxic stress and repressed by the transactivator. Binding of a small molecule regulator to the transactivator can, respectively, inhibit or enable its repressing function. Hence, the resulting gene switch is active in cells exposed to heat or proteotoxic stress in the presence or absence, respectively, of the small molecule regulator. The second component can also be a modified (or partial) heat shock promoter that requires co-activation by transactivator and endogenous HSF1. In this case, transactivator is activated by a bound small molecule regulator.

### BRIEF DESCRIPTION OF FIGURES

FIG. 1 presents a generic gene switch that can be activated by transient heat or proteotoxic stress in the absence or presence of a small molecule regulator.

FIG. 2 provides an example of the switch of FIG. 1, in which switch generic transactivator is replaced with mifepristone-activated activator GLVP.

FIG. 3 outlines how the switches of FIGS. 1 and 2 can be incorporated in the genome of a single adenovirus or the genomes of a pair of adenoviruses, respectively.

FIG. 4 A presents a generic gene switch that can be activated by transient heat or proteotoxic stress in the absence or presence of a small molecule regulator. The switch contains an auto-activation loop that causes transactivator expression to be maintained subsequent transient activation. Part B of the Figure shows an example switch, in which generic transactivator is replaced with mifepristone-activated activator GLVP.

FIG. 5 outlines how an example switch of FIG. 4 can be incorporated in the genome of a single adenovirus or the genomes of a pair of adenoviruses, respectively. Part C of the Figure also illustrates how a passenger gene can be introduced.

FIG. 6 generically outlines two types of alternative gene switches that can or may be used to regulate replication of viruses of the invention as well as passenger gene expression.

FIG. 7 provides the nucleotide sequence of pShuttle.

FIG. 8 provides the nucleotide sequence of pGene/V5-His.

FIG. 9 provides the nucleotide sequence of pXC1.

FIG. 10 provides the nucleotide sequence of pSwitch.

### DETAILED DESCRIPTION

Unless otherwise defined, all terms shall have their ordinary meaning in the relevant art. The following terms are defined:

“Replication of virus” or “virus/viral replication” is understood to mean multiplication of viral particles.

“Proteotoxic stress” is a physical or chemical insult that results in increased protein unfolding, reduces maturation of newly synthesized polypeptides or causes synthesis of proteins that are unable to fold properly.

A “small molecule regulator” is understood to be a low molecular weight ligand of a transactivator used in connection with this invention. Depending on the transactivator concerned, the small molecule regulator can inhibit or activate the transactivator. Small molecule regulator is typically, but not necessarily, smaller than 1000 Dalton (1 kDa).

To be able to use a single term for all regulatory proteins used in gene switches described herein, “transactivator” shall be understood hereinafter as being a DNA-binding protein, whose binding to a specific DNA sequence can affect, positively or negatively, transcription of a nearby gene. This definition includes traditional transactivators as well as proteins that interfere with transcription.

“Activated” when used in connection with a transactivated gene means that the rate of expression of the gene is at least one order of magnitude greater after activation than before activation. When used in connection with a transactivator, “active” or “activated” refers to a DNA-binding and transactivation-competent form of a transactivator in the case of a positively acting transactivator, and to a DNA-binding form of a transactivator in the case of a negatively acting transactivator.

“Promoter of a heat shock gene”, “heat shock gene promoter” and “heat shock promoter” are used synonymously.

Herein, a virus, whose genome includes a non-viral gene, is either referred to as a “virus” or a “viral vector”.

“Recombinant” refers to the presence of an alteration caused by an experimenter.

Preferred viruses of the present invention possess the engineered property that they only replicate when cells containing them are exposed to heat or another proteotoxic stress in the presence of a small molecule regulator. Replication of other preferred viruses can be triggered by heat or other proteotoxic stress in the absence of a small molecule regulator that represses replication. The viruses can further contain a passenger gene that may or may not be activated under the same conditions. Because heat can be directed, systemically present viruses of the invention can be activated locally, i.e., in a spatially restricted manner. The viruses of the invention may be used for research purposes. They may be introduced into the germline of an animal or may be introduced systemically or locally at any stage of development, including adult stage. After administration or withdrawal, respectively, of small molecule regulator, the experimental animal may be subjected to one or more courses of localized heat to activate virus in a particular tissue or group of cells, resulting in the ablation of the targeted cells and cells in surrounding tissue and/or in activation and local spreading of an activated passenger gene and, consequently, of the product of the passenger gene. Such experiments can assist in defining the function of particular tissues or groups of cells in an adult or developing animal or in elucidating the biological or therapeutic consequences of local expression of a passenger gene. The viruses of the invention will also be useful in cell-based therapies. Cells of heterologous origin may provoke a graft-vs-host response, in spite of immunosuppressive therapy. Conditionally replicating viruses present in these cells may be specifically activated in a tissue in which rejection is observed to achieve localized killing of offending cells while maintaining the benefit of the therapy in tissues in which no rejection is observed. In other applications, immune cells containing conditionally replicating virus of the invention may be administered as a tumor therapy. Immune cells reaching the tumor may be locally activated by heat in the systemic presence (or absence) of a small molecule regulator to trigger virus replication and cell lysis in the tumor tissue. Local cell lysis will provoke a local inflammatory response that should enhance development of specific immunity. Alternatively, cells may be infected with virus of the invention also including a therapeutic gene. After localized or systemic administration of the cells, virus replication may be activated in a desired tissue location to trigger virus replication and local dissemination of a therapeutic gene, avoiding, e.g., toxicity associated with systemic expression of the therapeutic protein. The viruses of the invention may also be used in experimental gene therapy using model animals and, eventually, humans. For example, a virus of the invention may be introduced into a tumor. Heat treatment in the systemic presence/absence of a small molecule regulator will initiate virus replication and tumor cell killing. The therapeutic efficacy of virus of the invention may be enhanced by the presence of a therapeutic gene, e.g., a passenger gene encoding a cytotoxic protein. Such virus will not only be induced to replicate in tumor tissue but also to express the toxic protein, resulting in increased anti-tumor activity. Alternatively, a virus of the invention may not by itself kill cells but may include a therapeutic gene, e.g., a passenger gene encoding a cytotoxic protein. Localized replication of the virus in a tumor will result in the dissemination of virus and therapeutic gene throughout the tumor. Tumor cells will be killed by the action of the therapeutic protein.

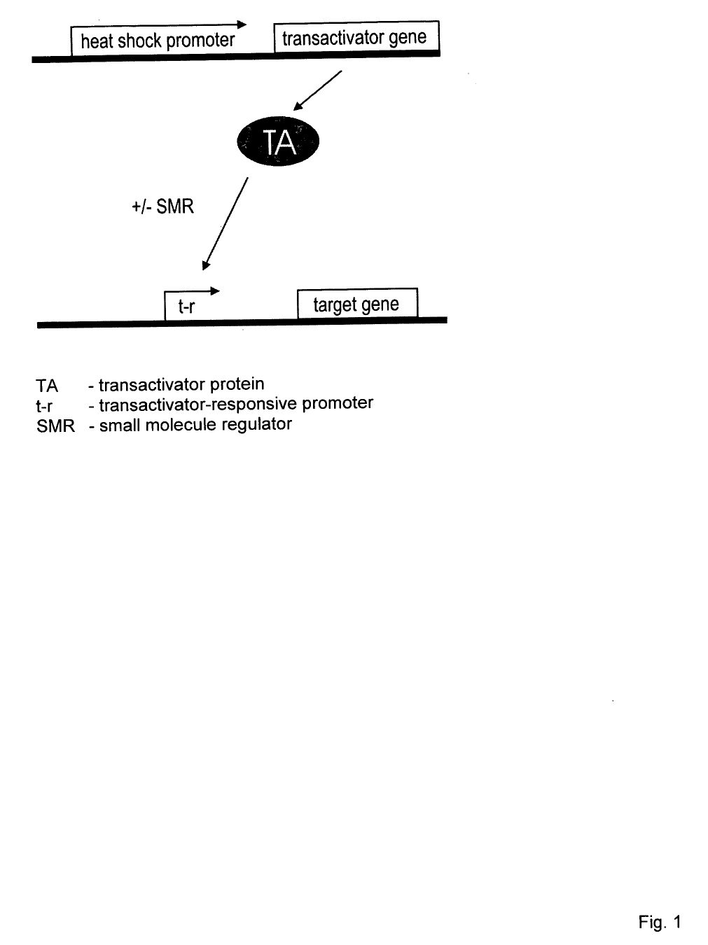

Replication of a virus of the invention is controlled by a gene switch that is activated by heat or other proteotoxic stress and the specific absence or presence of a small molecule regulator. Gene switches employed in the invention contain two components. Preferred gene switches used in connection with the conditionally replicating viruses of the invention comprise a gene for a small molecule-activated or small molecule-inhibited transactivator that is functionally linked to a promoter from a heat shock gene, and a promoter that is responsive to the transactivator. A gene to be regulated by the switch is functionally linked to the latter promoter that is responsive to the transactivator. In the case of adenovirus, the latter target gene may be the El gene. The operation of such a gene switch is outlined in FIG. 1.

A heat shock gene is defined herein as any gene, from any eukaryotic organism, whose activity is substantially enhanced when the cell containing the gene is exposed to a temperature above its normal growth temperature. Typically, the genes are activated when ambient temperature is raised by 3-10° C. Heat shock genes comprise genes for the “classical” heat shock proteins, i.e., Hsp110, Hsp90, Hsp70, Hsp60, Hsp40, and Hsp20-30. They also include other heat-inducible genes such as genes for MDR1, ubiquitin, FKBP52, hemoxidase and other proteins. The promoters of these genes, the “heat shock promoters”, contain characteristic sequence elements referred to as heat shock elements (HSE) that consist of perfect or imperfect sequence modules of the type NGAAN or AGAAN, which modules are arranged in alternating orientations (Xiao, H, and Lis, J. T. 1988. Science 239, 1139-1142; Amin, J., Ananthan, J., and Voellmy, R. 1988. Mol. Cell. Biol 8, 3761-3769; Fernandes, M., Xiao, H., and Lis, J. T. 1994. Nucleic Acids Res. 22, 167-173). These elements are highly conserved in all eukaryotic cells such that, e.g., a heat shock promoter from a fruit fly is functional and heat-regulated in a frog cell (Voellmy, R., and Rungger, D. 1982. Proc. Natl. Acad. Sci. USA 79, 1776-1780). HSE sequences are binding sites for heat shock transcription factors (HSFs; reviewed in Wu, C. 1995. Annu. Rev. Cell Dev. Biol. 11, 441-469). The factor responsible for activation of heat shock genes in vertebrate cells exposed to heat or a proteotoxic stress is HSF1 (Baler, R., Dahl, G., and Voellmy, R. 1993. Mol. Cell. Biol. 13, 2486-2496; McMillan, D. R., Xiao, X., Shao, L., Graves, K., and Benjamin, I. J. 1998. J. Biol. Chem. 273, 7523-7528). Preferred promoters are those from inducible hsp70 genes. A particularly preferred heat shock promoter used in the viruses of the present invention is the promoter of the human hsp70B gene (Voellmy, R., Ahmed, A., Schiller, P., Bromley, P., and Rungger, D. 1985. Proc. Natl. Acad. Sci. USA 82,4949-4953).

Examples for small molecule-regulated transactivators than can be incorporated in the gene switches used in viruses of the invention include tetracycline/doxocycline-regulated tet-on and tet-off repressors (Gossen, M. and Bujard, H. 1992. Proc. Natl. Acad. Sci. USA 89, 5547-5551; Gossen, M., Freundlieb, S., Bender, G., Muller, G., Hillen, W. and Bujard, H. 1996. Science 268, 1766-1769), ecdysterone/ponasterone-regulated insect steroid receptor-based transcription factor EcR/RXR (No, D., Yao, T. P., and Evans, R. M. 1996. Proc. Natl. Acad. Sci. USA 93, 3346-3351), mifepristone-regulated GAL4-steroid receptor-activation domain chimeras such as GLVP (Wang, Y., DeMayo, F. J., Tsai, S. Y., and O'Malley, B. W. 1997. Nat. Biotechnol. 15, 239-243; Wang, Y., Xu, J., Pierson, T., O'Malley, B. W., and Tsai, S. Y. 1997. Gene Ther. 4,432-441), versions of which transactivator are now also commercialized under the name “GeneSwitch” (Invitrogen Corporation, Carlsbad, Calif.), and rapamycin-regulated chimeric factor NFκB-FRB/FKBP-ZFHD1 (Rivera, V. M., Clackson, T., Natesan, S., Pollock, R., Amara, J. F., Keenan, T., Magari, S. R., Philips, T., Courage, N. L., Cerasoli, F. Jr., Holt, D. A., and Gilman, M. 1996. Nat. Med. 2, 1028-1032; Magari, S. R., Rivera, V. M., Iuliucci, J. D., Gilman, M., and Cerasoli, F., jr. 1997. J. Clin. Invest. 100, 2865-2872). Other small molecule-regulatable transactivators may be used, provided that they can be employed to control the activity of a target gene without also causing widespread deregulation of genes of the host cell and provided further that the associated small molecule regulators have a sufficiently low toxicity for the host cell at their effective concentrations.

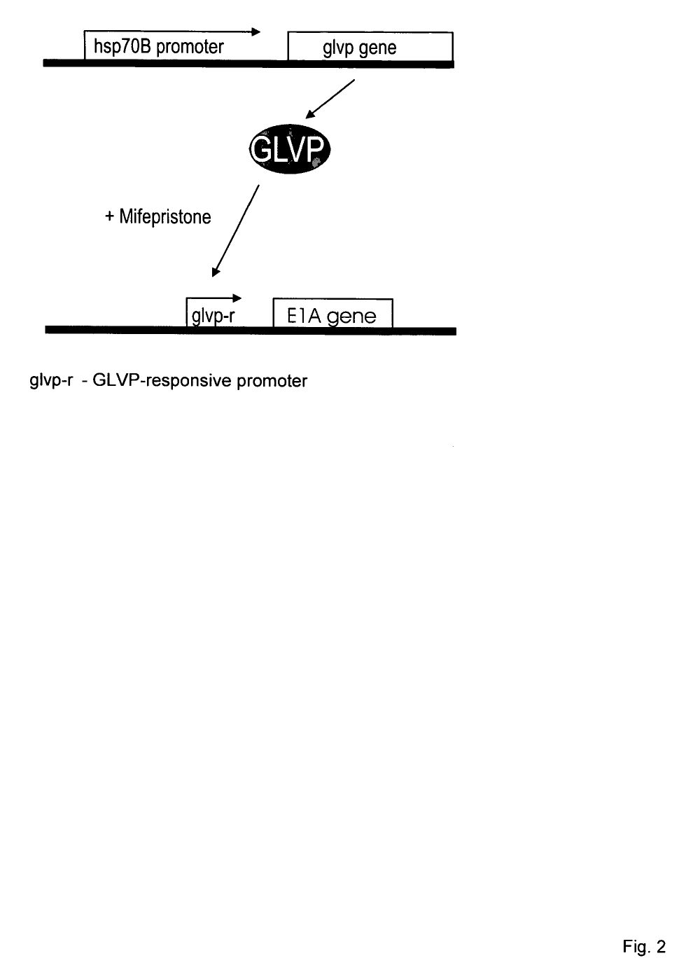

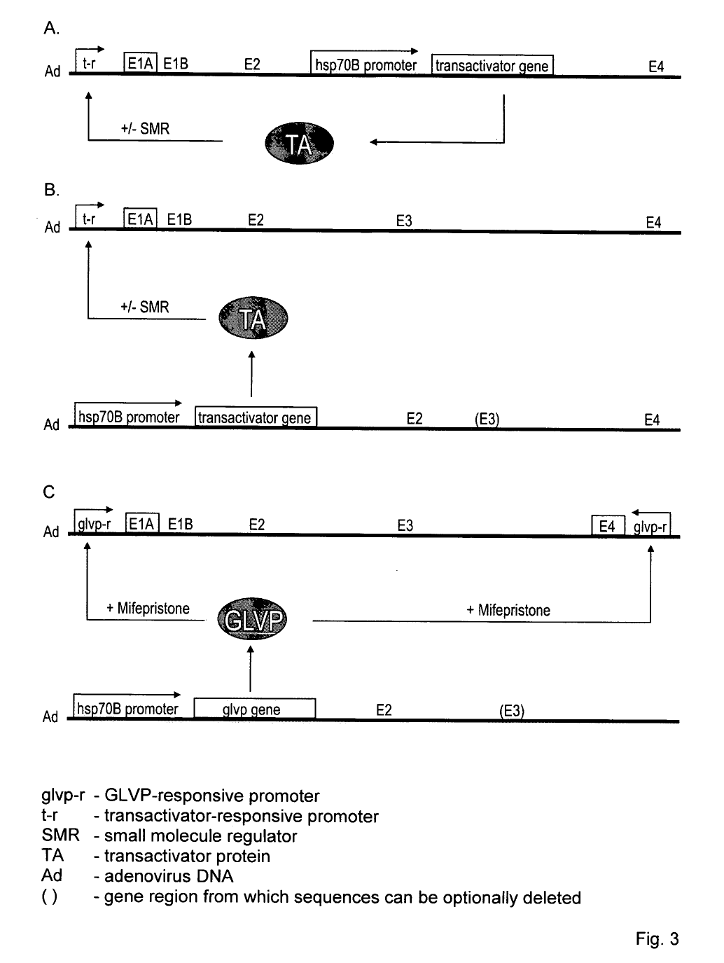

In the example in FIG. 2, a gene for mifepristone-activatable, chimeric transactivator GLVP is functionally linked to strictly heat-regulated human hsp70B heat shock promoter. The target gene is a gene required for viral replication such as, in this example, the E1A gene of adenovirus. The target gene is functionally linked to a promoter that is responsive to the transactivator. In the present example, a suitable promoter will be a minimal promoter supplemented with binding sites for GAL4. A gene switch of the kind outlined in FIG. 1 may be incorporated in a viral vector as shown in FIG. 3A. In this example, a transactivator-responsive promoter replaces the E1A promoter of adenovirus, and transactivator gene cassette replaces a non-essential gene segment of the virus, e.g., an E3 region in adenovirus. Alternatively, or if there is insufficient room in the deleted virus genome for inclusion of a chosen transactivator gene, the gene switch may be distributed between two viral vectors. In the example in FIG. 3B, a transactivator-responsive promoter replaces the E1A promoter of an otherwise wildtype adenovirus genome, and hsp70B-directed transactivator gene is inserted into the genome of a typical replication-deficient adenovirus lacking E1 and, possibly, E3 sequences. A specific example pair of conditionally replicating viruses of the invention is shown in FIG. 3C. The first adenovirus is wildtype, except for E1A and E4 promoter regions that are replaced by transactivator-responsive promoters. The second virus is a replication-deficient virus that minimally lacks E1A and E4 functions. A hsp70B promoter—glvp gene cassette is inserted in the genome of the latter virus.

In order for viral replication to occur, cells will need to be co-infected with the recombinant virus pair. Gene switch-directed virus replication would be effected as follows: subsequent to infection of a cell with the recombinant virus combination of FIG. 3C, the cell would be exposed to an appropriate concentration of small molecule regulator mifepristone and subjected to a transient heat treatment to activate the hsp70B promoter. GLVP will be expressed and be activated by mifepristone. Active GLVP will then transactivate E1A and E4 gene expression from the first virus genome, resulting in replication of the virus pair. Cells in which the adenoviral replication occurred will lyse, virus particles will be liberated, and adjacent cells will be infected. Upon administration of a further transient heat treatment, replication in the secondarily infected cells will be triggered, and so forth.

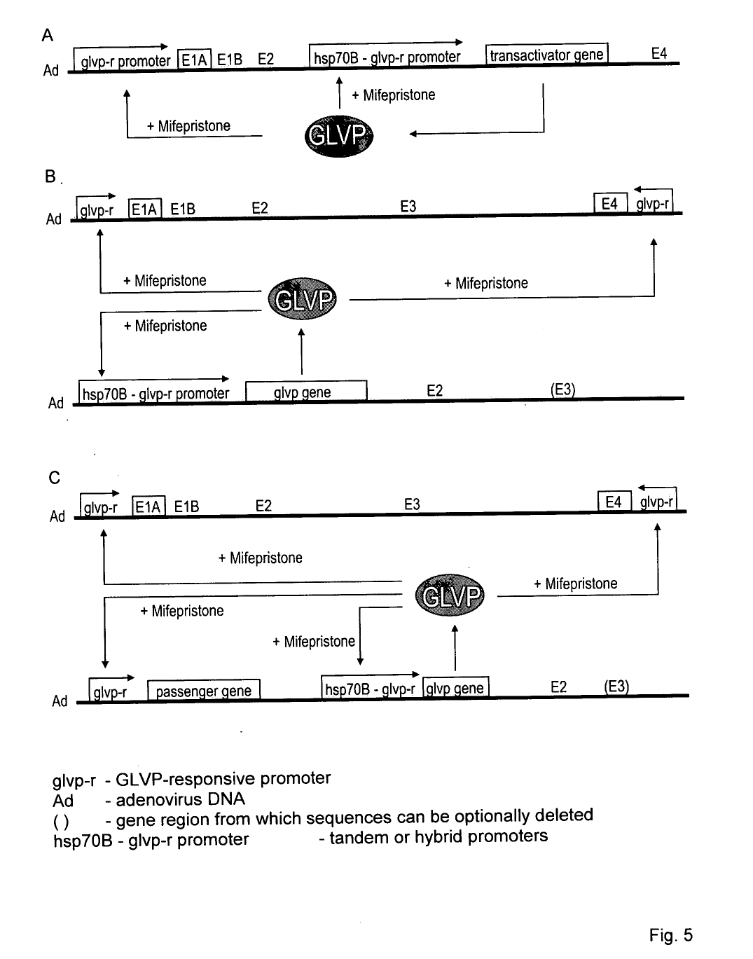

A virus or virus pair of the invention may also include a passenger gene. Whether the passenger gene or even a transactivator gene can be incorporated into a viral genome containing all genes and other sequences required for replication will depend on the type of virus, the maximal genome size for packaging, knowledge about non-essential viral genes that can be deleted, and the sizes of transactivator and passenger genes to be inserted. In the case of adenovirus, it may be necessary to insert the passenger gene and, possibly, even the transactivator gene into a partially deleted or completely gutted viral genome. As illustrated by the example case of FIG. 5C, the “virus of the invention” will be a pair of viruses, the first of which will contain a complete genome except for a substitution of E1A and E4 promoters with GAL4-binding site-containing glvp-r promoters that are responsive to GLVP transactivator. The second virus will be replication-deficient. Space provided by sizeable deletions of viral genes, typically E1 and E3 gene regions (and in this particular case also E4 sequences), will be taken by a passenger gene and a transactivator gene. The transactivator gene will be under the control of a functionally linked heat shock promoter, or the hybrid or tandem hsp70B-glvp-r promoter actually shown in the Figure (and discussed below). The passenger gene may be linked to a glvp-r promoter or another promoter, as the application may require. It is noted that when a pair of viruses is used, the viruses are typically of the same species. The underlying reason is that typically at least one virus of the virus pair is dependent for replication on species-specific viral proteins expressed from genes carried by the other virus. It is further noted that it is not necessary that any one of the two viruses contain all genes required for replication. These genes may be distributed over both viruses. What is important is that, together, the two viral genomes encode all required viral functions.

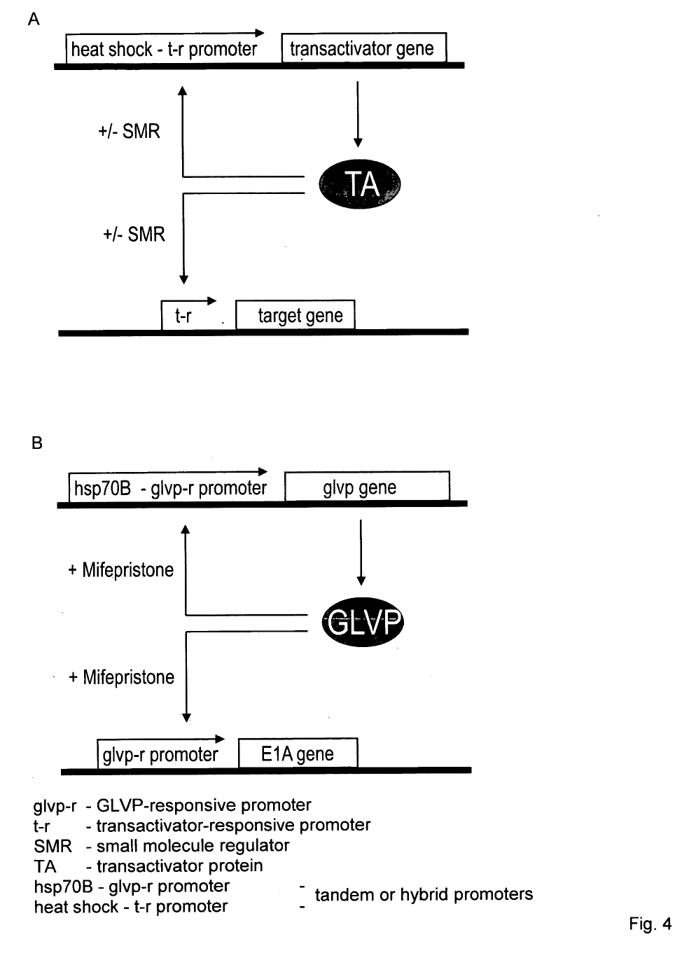

Most preferably, replication of viruses of the invention and, if desired, of passenger gene, is controlled by more complex switches described in U.S. Pat. No. 6,342,596 and in U.S. patent application Ser. No. 10/046420. Unlike the above-described switches, these switches contain a auto-activating element that allows them to remain active long after an activating heat treatment or exposure to other proteotoxic stress. A generic outline of the components and operation of such switches is shown in FIG. 4A. A switch of this kind also comprises two components. The first component is a gene for a small molecule-regulated transactivator, which gene is functionally linked to a DNA sequence that acts as a heat shock promoter as well as a transactivator-responsive promoter. This DNA sequence may contain a hybrid promoter comprising binding sites for both HSF1 and transactivator. Alternatively, it may contain two separate promoters, one of them a heat shock promoter and the other a transactivator-responsive promoter, provided that each of the promoters can direct expression from the linked transactivator gene. The second component is a target gene that is functionally linked to a transactivator-responsive promoter. When a cell containing the two components of such a switch is exposed to transient heat or other proteotoxic stress, HSF1 is activated and induces transcription from the transactivator gene. Depending on whether the particular transactivator used is activated or inhibited by a small molecule regulator, in the presence of absence of its small molecule regulator, newly made transactivator will activate the expression of the target gene as well as promote expression of additional transactivator protein. As a consequence, transactivator level is maintained after HSF1 has been inactivated subsequent to the transient stress and target gene expression continues. Withdrawal or addition, respectively, of small molecule regulator will result in the inactivation of the switch. FIG. 4B shows an example a switch of this kind, in which the transactivator is mifepristone-activated transactivator GLVP and the target gene is the E1A gene of an adenovirus. In FIG. 5, the incorporation of the latter example switch in adenoviruses of the invention is illustrated. FIG. 5A illustrates how the glvp gene and the linked hybrid or tandem promoter are introduced into the E3 region subsequent to deletion of sequences from this region. The E1A promoter is replaced by GAL4-binding site-containing promoter glvp-r that is responsive to GLVP. Whether such a recombinant can actually be made depends on the virus' packaging limit and on the lengths of the deleted and inserted sequences, respectively. In the example in FIG. 5B, the switch is distributed over a pair of viruses. One of these viruses may contain a complete viral genome, except for replacement of the E1A and E4 promoters with glvp-r promoters. The other virus is a replication-deficient virus that may lack E1, E3 and E4 sequences, but contain the glvp gene and the linked hybrid or tandem promoter that is activatable by heat and active GLVP. As shown in the graph in FIG. 5C, a passenger gene may also be included in the defective genome of the latter virus. The passenger gene may be controlled by a transactivator-responsive promoter or another promoter.

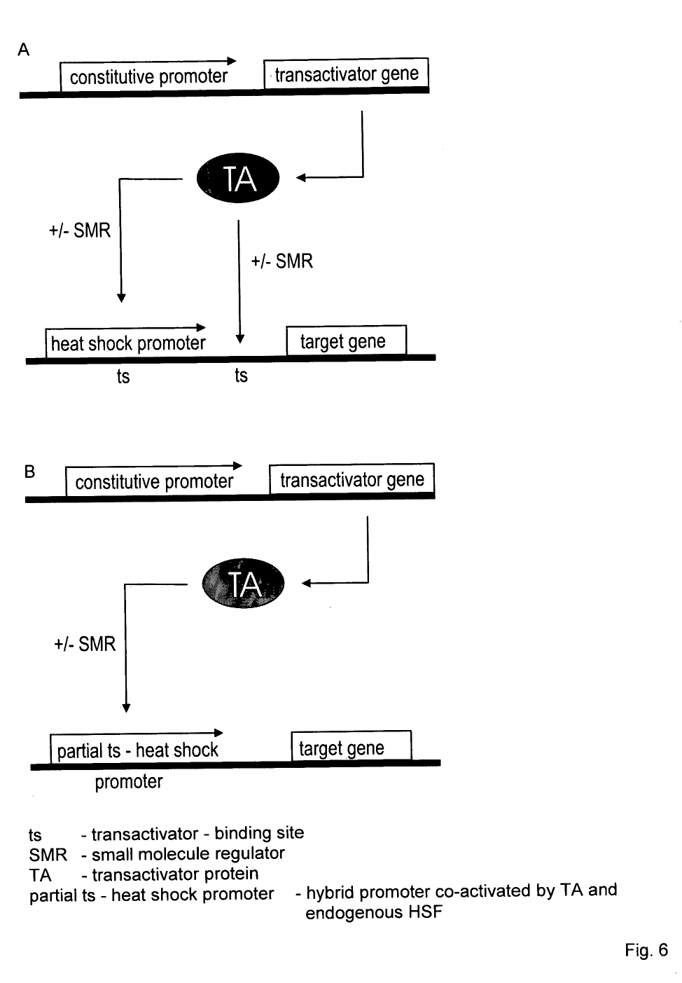

Alternatively, replication of a virus or virus pair of the invention and, if desired, expression of a passenger gene can be controlled by a type of gene switch (FIG. 6A) comprising a constitutively expressed gene for a transactivator (that is not transactivation-competent), whose DNA-binding activity is regulated by binding of a small molecule regulator, and a modified heat shock promoter, including 5′ untranslated sequences, that contains one or more binding sites for the transactivator. The latter promoter is functionally linked to a viral or passenger gene to be regulated. Transactivator binding sites are so placed in the heat shock promoter (and/or RNA leader region) that binding of transactivator prevents heat activated transcription of the linked gene. Binding of transactivator to sites inserted near HSF1-binding sites may prevent HSF1 binding to the promoter; binding of transactivator to sites inserted in the 5′ untranslated sequences may inhibit transcription elongation. The resulting gene switch is activated by transient heat in the presence or absence of small molecule regulator. An additional type of gene switch (FIG. 6B) that may be used to control replication of viruses of the invention and, if desired, expression of a passenger gene comprises a constitutively expressed gene for a transactivator, whose DNA-binding activity is activated or inhibited by binding of a small molecule regulator, and a modified heat shock promoter including binding sites for the transactivator. These binding sites, that may be low-affinity binding sites for the transactivator, or, possibly, half sites, are insufficient to allow activation of the promoter by the transactivator alone. The promoter is effectively co-activated by HSF1 induced by a transient heat or other proteotoxic stress and active transactivator. A modified heat shock promoter that requires co-activation may be obtained by deletion of all but one heat shock elements in a heat shock promoter, whose activation depends on the presence of multiple heat shock elements, and addition of one or more transactivator-binding sites.

Viruses particularly suitable for use in the present invention are those that were previously used as replicating or oncolytic viral vectors such as adenoviruses, herpesviruses, and retroviruses, including foamy viruses.

The biology and genetics of adenovirus are well understood (Russell, W. C. 2000. J. Gen. Virol. 81, 2573-2604; Hitt, M. M., Addison, C. L., and Graham, F. L. 1997. Advances in Pharmacology 40, 137-206). Persons skilled in the art know how to prepare and administer replication-competent or replication-deficient adenovirus vectors. Strategies used to control replication of adenovirus were recently reviewed (Post, D. E., Khuri, F. R., Simons, J. W., and Van Meir, E. G. 2003. Hum. Gene Ther. 14, 933-946). Typically, conditional replication of adenovirus was achieved by replacing the E1A, E1B or E4 viral promoter with a regulated or tissue-selective promoter of choice. In some cases, two of the latter viral promoters were replaced. For example, Hernandez-Alcoceba et al. (Hum. Gene Ther. 11, 2009-2024 (2000)) prepared a conditionally replicating type 5 adenovirus by replacement of the E1A and E4 promoters by truncated pS2 promoters containing binding sites for estrogen receptor. The recombinant virus replicated and lysed efficiently estrogen receptor-positive but not estrogen-receptor-negative cells. Yu et al. (Cancer Res. 59, 1498-1504 (1999)) constructed conditionally replicating adenovirus 764 by substituting the E1A and E1B promoters with a PSE (prostate-specific enhancer) promoter and a promoter fragment from a kallikrein 2 gene, respectively. Virus 764 efficiently replicated in and killed cells from a prostate tumor cell line but not cells from several other cell lines.

Herpesviruses are enveloped, double-stranded DNA viruses with large genomes (152 kbp for HSV-1). This large genome size had hampered genetic analyses and experiments in the past. This problem has been solved by the use of bacterial artificial chromosomes (BACs) that allow integration of an entire herpesvirus genome, enabling the use of efficient mutagenesis and recombination protocols to manipulate the viral genome (Wagner, M., Ruzsics, Z., and Koszinowski, U. H. 2002. Trends in Microbiol. 10, 318-324). The products of at least two of the immediate early genes of herpesvirus, ICP4 and ICP27 are essential for viral replication (Burton, E. A., Bai, Q., Goins, W. F., and Glorioso, J. 2002. Curr. Opin. Biotechnol. 13, 424-428, and references cited therein). Manipulations that place one of these genes under the control of a regulated or tissue-selective promoter will produce a conditionally replicating herpesvirus. For example, Miyatake et al. (J. Virol. 71, 5124-5132 (1997)) made use of HSV-1 mutant d120 that lacked both copies of the a4 gene, the gene encoding ICP4, and inserted into this mutant an a4 gene copy that was functionally linked to a promoter containing an albumin enhancer and promoter element. The resulting recombinant herpesvirus replicated three log scales better in albumin-expressing cells than in other cells. Furthermore, the virus effectively inhibited growth of an albumin-expressing tumor xenograft. Herpesviruses can be made to preferentially replicate in mitotically active cells by deletion of genes for thymidine kinase, ICP6 or ICP34.5 (Harrington, K. J., Bateman, A. R., Melcher, A. A., Ahmed, A., and Vile, R. G. 2002. Clinical Oncology 14, 3-16, and references cited therein).

Retroviruses are single-stranded, diploid RNA viruses that have been intensively investigated and used as replication-defective vectors to transfer therapeutic genes (Vile, R. G., and Russell, S. J. 1995. Br. Med. Bull. 51, 12-30). Persons skilled in the art know how to manipulate retroviral genome and how to manufacture retroviral vectors. Preparation of retroviral vectors and their uses are described in many publications including European patent application EP 0178220; U.S. Pat. No. 4,405,712; Gilboa. 1986. Biotechniques 4, 504-512; Mann et al. 1983. Cell 33,153-159; Cone and Mulligan. 1984. Proc. Natl. Acad. Sci. USA 81, 6349-6353; Eglitis, M. A. et al. 1988. Biotechniques 6, 608-614; Miller, A. D. et al.1989. Biotechniques 7, 981-990; Miller, A. D. 1992. Curr. Top. Microbiol. Immunol. 158, 1-24; PCT application Ser. No. WO 92/07943 entitled “Retroviral Vectors Useful in Gene Therapy”; Hu, W. S., and Pathak, V. K. 2000. Pharmacol. Rev. 52, 493-511. Promoters driving the expression of viral genes reside in the U5 and U3 regions of provirus. The actual viral genome lacks the U5 region. Hence, it only contains the promoter of the U3 region. Recently, Logg et al. (J. Virol. 76, 12783-12791 (2002)) demonstrated that replacement of the promiscuous transcriptional control elements within the U3 region in murine leukemia virus with sequences from the promoter of the androgen-regulated probasin gene that is active in the prostate, or from a variant promoter derived from the probasin promoter, resulted in recombinant virus that preferentially replicated in prostate-derived cells. Nestler et al. (Gene Ther. 4, 1270-1277 (1997) reported the construction of a replication-competent foamy virus that lacked a large segment of the U3 region and most of the bet gene. Genes encoding prodrug-activating enzymes, i.e., herpes thymidine kinase, cytosine deaminase and polynucleoside phosphorylase were fused to the truncated bet gene. The latter “suicide” proteins were expressed as fusion proteins, which were processed to yield unfused proteins due to the presence of a self-cleaving sequence at the fusion point.

More generally, viruses suitable for use in the invention include retroviruses, and single-stranded and double-stranded DNA viruses that utilize the host transcriptional machinery and whose replication depends on proteins expressed post infection. The latter DNA viruses include the Hepadnaviridae, Parvoviridae, Papovaviridae, Circoviridae, Adenoviridae and Herpesviridae families.

Papovaviridae include at least nine human papilloma viruses and polyomavirus BK and JC.

Retroviridae include those of genera Lentivirus, Spornavirus and others such as HTLV-I, HTLV-II, HIV-I, HIV-II, bovine leukosis virus, feline sarcoma and leukemia viruses, avian reticuloendotheliosis, mouse mammary tumor virus, visna viruses, equine infectious anemia virus, FIV, bovine lentivirus, SUV, and foamy virus.

Hepadnaviridae include hepatitis B-like viruses, e.g. hepatitis B and other related viruses.

Parvoviridae includes those of genera parvovirus, dependovirus and densovirus, such as human and animal parvoviruses.

Adenoviridae include those of genera Mastadenovirus and Aviadenovirus, such human and animal mastadenovirus strains.

Herpesviridae include those of genera Simplexvirus, Varicellavirus, Betaherpesvirinae, Cytomegalovirus, lymphocryptovirus, Marek's disease-like viruses and Rhadinovirus. Examples are herpes simplex, varicella-zoster virus, human cytomegalovirus, Marek's disease virus, EB virus and others.

Circoviridae include the genus Circovirus. Example circoviruses are chicken anemia virus, psittacine beak and feather disease virus, and porcine circovirus.

Extensive descriptions of the biology, biochemistry and genetics of the above virus families and their known members are found in “Virology” (B. N. Fields, D. M. Knipe, and P. M. Howley, eds.), vol. 1 and 2, Lippincott-Raven Publishers, Philadelphia, Pa. (1996).

Passenger genes may include cell-affecting genes such as genes that encode apoptopic inducers, genes that affect cell death, aging, division and DNA synthesis, mitochrondial genes, peroxisomal genes, immune response-related genes, ATP-binding proteins, cytoskeletal genes, all rescue genes, genes involved in cell damage and repair. A listing of potential yeast and mammalian genes which may be included in the viral vectors of the invention is provided below.

Yeast Genes:

CELL RESCUE, DEFENSE, CELL DEATH AND AGING PRE3, PRE1, PUP2, RPN12, RPT1, MAG1, OGG1, SED1, ATH1, SPE2, GRE3, TPS2, TPS1, ATR1, ATX1, SK13, SK12, SK18, APN1, HPR5, ERG5, CCZ1, SRA1, SNF1, YCK1, YCK2, HRR25, CTA1, CTT1, WSC4, PAM1, TIR2, TIR1, HDF2, TFB4, RAD1, HAM1, LYS7, SOD1, KIN28, DIT2, ERG11, CYC7, CCP1, PHR1, DAK2, DAK1, ALR1, ALR2, HOR2, RAD17, DDC1, DDR2, ALK1, HEL1, SSL2, RAD5, SGS1, PIF1, RAD3, CDC9, REV7, NTG1, RAD18, RAD57, RAD55, XRS2, RAD30, MMS21, RAD51, RAD10, PS02, REV1, DIN7, RAD54, CDC2, PES4, POL2, REV3, RPB7, RPB4, SGE1, UBA1, UBC4, UBC5, RAD6, QR18, RNC1, NTG2, ERC1, RAD4, ETH1, FKB2, YHB1, FLR1, MEC3, ZWF1, GSH1, GRX1, TTR1, HYR1, GLR1, YCF1, FPS1, GPD1, RAS2, RAS1, CUPS, HSP26, HSP30, HSP12, HSP104, DDR48, HSC82, HSP82, MDJ1, MDJ2, HSP60, HSP78, ECM10, SSE1, SSA1, SSA3, SSA4, SSA2, SSE2, HSF1, HIG1, HDF1, HMS2, GRE1, DD11, RTA1, SIMI, LAG2, ZDS1, MET18, SNG1, NCA3, KTI12, UTH1, SUN4, SSU81, SSD1, TH14, KAR3, LIF1, SFA1, LAG1, LTV1, MDR1, SSK22, SSK2, HOL1, CIS3, HSP150, PIR3, MAC1, CUP1 A, CUP1 B, YDJ1, SSQ1, SSC1, IMP2, MPT5, ATX2, SN02, MLP1, NHX1, NCP1, NSR1, SNF4, RAD16, RAD7, RAD14, RAD23, ROD1, MGT1, OSM1, SIP18, SAT2, MNR2, MMS2, PNT1, CYP2, PAD1, PDR5, PDR3, PDR6, RTS1, PA13, HOR7, DUN1, IRE1, MKK2, MET22, PPZ2, PTC1, PTP2, MMS4, RAD52, PDR13 SLG1, GRR1 HIT1, RDH54 BR01, PIR1 MSRA, RNR4 RNR3, HAL1, YGP1, CDC55, PPZ1, PKC1, HAL5, MKK1, HOG1, SLT2, BCKi, RAD53, SIR4, SIR3, SIR2, MGA1, FUN30, YR02, DNL4, RRD1, SAT4, RAD27, MSN2, ST11, PAU3, PAU2, PAU5, PAU1, PAU4, PAU6, (MLP1), RAD2, FZ_F1, SSU1, SOD2, CRS5, BCK2, ASM4, TIP1, TFB1, CCL1, SSL1, TFB3, TFB2, TSAI, TRX1, TRX2, ROX3, PDR1, GTS1, MCM1, SKN7, CAD 1, MSN4, YAP1, SLN1, SSK1, PBS2, UB14, RSP5, SVS1, ZRC1

CELL GROWTH, CELL DIVISION AND DNA SYNTHESIS GSC2, PLC1, PRE3, PRE2, PRE1, PUP2, RPN12, RPT6, RPT1, DIS3, RP SOA, AGA1, AGA2, ASG7, ACH1, ACT1, SAC6, ARP100, ABP1, PAN1, ARP 2, AREIARE2, SPE2, CYR1, SRV2, ADK2, GCS1, SOH1, TUB1, TUB3 SAG1, AKR1, YAR1, SK18,

ARG82, ABF1, STE6, BAR1, 8011, TUB2, RBI-2, BIG1, BI M1, BAT1, BEM1, BEM4, SBE2, BN14, BUD6, 8012, BUD9, BUD4, BUD8, RC K2, CMK1, CNA1, CMP2, CNB1, CCH1, CMD1, SRA1, YCK1, YCK2, HRR2 5, CKA2, CKA1, YCK3, EST2, TFS1, SCM4, GIC2, GIC1, CAK1, BUB2, B UB3, ESR1, RAD24, DBF20, PDS1, HPC2, NUD1, CDC47, CDC10, CDC13, C DC37, CDC1, CDC40, CDC4, CDC20, CDC6, CDC46, CDC3, KAR1, BB P1, CDC50, FUS1, KRE9, EGT2, ARP1, CHS1, CHS2, CHS3, CHS5, MS11, CAC2, R LF2, CHL4, SMC1, SMC2, CIN1, SNF7, CLC1, COF1, PAM1, LAS17, HDF2, SEC3, SNF2, SWI1, SNF5, SNF1 1, DOC1, APC2, APC5, TAP42, CDC53, KAR 9, CCE1, CLB6, CLB5, CLN3, PCL2, CLN1, PCL1, CLN2, CI-133, CI-131, CLB 4, CI-132, FAR1, CKS1, CDC28, PH085, KIN28, SSN3, CLG1, DIT2, SLA1, SLA2, SP020, DPP1, RAD17, DDC1, HEL1, DNA2, RAD5, SGS1, HCS 1, PIF1, CDC9, MSH3, MSH6, MLH1, PMS1, MSH2, MSH1, POL4, REV 7, MRE11, RAD26, RAD9, RAD18, RAD57, RAD55, XRS2, MMS21, RAD51, RADIO, RAD 50, RFA3, RFA2, RFA1, RFC4, RFC5, RFC3, RFC2, RFC1, FOB1, TOP1, TO P2, TOP3, RAP1, RAD54, PR12, PR11, POL1, POI-12, CTF4, HUS2, CDC2, P ES4, POL2, DPB2, DPB3, MIP1, REV3, SSN8, GAL11, RGR1, SRB6, RP041, SEC59, DIP2, CDC14, MSG5, DYN1, UBC4, UBC9, CDC34, UBC5, UBC 1, UBC6, RAD6, QR18, ELC1, RNC1, CTS1, KEX2, APG1, SSP1, SUP35, EXM2, S PR1, EXG1, EXG2, DHS1, CAP1, CAP2, BRN1, GPR1, GIF1, MEC3, TU B4, CIS2, LTE1, SDC25, SRM1, CDC25, ROM2, BUDS, ROM1, SPT16, CDC43, G IP1, SIN4, SNF6, KRE6, GFA1, NGR1, WH12, RSR1, CIN4, RAS2, RAS1, GP A1, STE4, STE18, CDC42, MDG1, SEC4, TEM1, RH03, RH04, RI-102, RHO 1, CDC24, BEM2, BUD2, BEM3, LRG1, GPA2, SIS1, HSP82, HSF1, ABF2, HDF1, HDR1, RPD3, HSL7, HO, SBA1, HPR1, IDS2, NFI1, CSE2, MDM 1, MI-1 131, MIDI, SIMI, HIR3, SIS2, MAKI 1, LAS1, SPA2, WH14, ECM33, SET1, CTF19, CIN2, MCM16, SLK19, CYK2, CNM67, SST2, DPB11, DOS2, D FG16, AFR1, ZDS1, SR07, PEA2, FAR3, SMP2, WH13, CDC5, MET30, SAS2, SCC2, CIS 1, STN1, UTH1, PAC2, SSD1, SRP1, KRE5, KIP1, CIN8, SMY1, KIP2, KAR3, KIP3, CBF1, CBF2, SKP1, CEP3, CTF13, DBR1, LAG1, MIH1, BFR1, DIG2, DIG1, MFA1, MFA2, MFAlpha1, MFAIpha2, MID2, SSF1, MATALPHA2, MATA LPHA1, ALPHAI, ALPHA2, A2, A1, SAN1, PGD1, SPO11, MSH5, DMC 1, ISC10, MSH4, SP013, NDT80, REC104, HOPI, RED1, SP07, MUM2,ME15, S AE2, NAM8, REC107, REC102, REC114, MER1, RIM01, NDJ1, CDC54, CP R7, SYG1,MCM2, CIS3, HSP150, ACE2, CDC48, ASE1, YTM1, HSM3, YD J1, ERV1, FUS3, JNM1, MCD1, MMC1, MSB1, MSB2, MPT5, ZDS2, MSN5, KEM1, MLC1, MY02, MY04, MY05, MY03, MY01, DEC1, PMD1, M DS3, ASH1, UME1, UME6, NHP6A, RFT1, TRF5, NNF1, NDC1, BIK1, KAR2, KAR5, NUM1, CDC39, MAK16, NAP1, RAD16, RAD23, NBP35, ORC1, ORC6, ORC5, ORC4, ORC3, RRR1, SIC 1, BUD3, PWP2, STE3, STE2, OPY2, STE50, STE5, PEL1, TOR1, TOR2, PIK1, STT4, MSS4, SP014, POL32, IME4, SHP1, PDS5, FEN1, CSE1, FLO8, PFY1, PHB2, PHB1, POI-30, AXL1, STE23, RAD28, CDC7, SMP3, MKK2, CDC15, ARD1, CHU, PPH3, PPH21, PPH22, PTC1, SE C9, PPS1, PTP3, YVH1, PTP2, PUS4, PCH2, PCH1, CBF5, SEF1, MMS4, SHR5, RAD59, RAD52, RHC18, RGP1, RVS167, RIM9, BNR11, BN11, SPT3, SOK2, KAR 4, DBF4, SDS22, MCM3, CTF18, SR04, SPH1, FUS2, MOB1, FL08, FIG1, FIG2, END3, DFG5, CTR9, TOM1, POP2, GRR1, SCP160, SUR1, MUM3, ZIP2 CDC45, RDH54, SHE3, SHE2, SHE4, GPI1, MIF2, ESP1, HOP2, DNA43, SMC3, PAC11, PAC10, RD11, RGA1, RNR1, RNR2, RNR4, RNR3, PRPS 1, RPL10, RPS1A, MTF1, SN12, CDC12, CDC11, SPR28, CDC55, GLC7, PKC1, GI N4, SPS1, RCK1, BUB1, IME2, YAK1, YPK2, RIM11, CLA4, MKK1, MEK1, I PL1, SGV1, SLT2, KSS1, BCK1, STE11, STE20, DBF2, HSL1, NRK1, SIT4, T PD3, ELM1, MCK1, RAD53, STE7, SWE1, MPS1, SAS3, HST1, SIR4, SIR3, SIR1, SIR2, CTH1, DOM34, HST4, RVS161, DNL4, IQG1, FUN16, HYM1, RT S2, MNN10, PRK1, MCM6, SAP155, SAP4, SAP190, SAP185, MUD13, M AD1, CIK1, NUF1, SPC97, SPC42, SPC98, CDC31, NUF2, MAD3, MAD2, DIT1, YSW1, SP012, SP016, MCD4, BDF1, SGA1, GSG1, SHC1, CDA1, CD A2, SMK1, SPS2, SPR6, SLZ1, SPS4, SPR3, SPS100, SPS18, RAD27, SNZ 1, SUR4, ST11, SBE22, CSE4, BMH 1, SVL3, SCH9, (MLP1), SSF2, RAD2, CDH1, CDC27, CDC26, CDC23, CDC16, APC1, APC 11, APC4, APC9, SAP30, RSC6, RSC8, STH1, SFH1, SAS5, JSN1, BMH2, SMT4, BCK2, HOC1, ZIP1, UFE1, EST1, TEL1, ANC1, CCL1, DST1, TRX1, TRX2, TRF4, PAT1, SPT4, SP T6, CDC36, SWI5, SWI4, PHD1, SWI6, GTS1, MCM1, IME1, SKN7, MBP1, SW13, SIN3, STE12, CIN5, SDS3, SP01, MOT2, RPG1, PRT1, CDC33, TPM1, TPM2, TWF1, TEC1, TTP1, STE13, PRP8, UB14, DSK2, RSP5, D OA4, UNG1, VPS45, VAN1, VRP1, DFG10, YHM2, GL′Q3, SFP1, STE24, RME1, SAE3, ME14, NHP6B, MOB2, EST3, RIM1

HEAT SHOCK PROTEINS CAT5, CPH1, CTT1, CYP2, DDR2, FPR2, HSC82, HSP104, HSP12, HSP150, HSP26, HSP30, HSP42, HSP60, HSP78, HSP82, KAR2, MDJ1, SIS1, S OD2, SSA1, SSA2, SSA3, SSA4, SSB1, SSB2, SSC1, SSE1, SSE2, ST11, TIP 1, TPS2, UB14, YDJ1

MITOCHONDRIAL AAC1, AAC3, AAT1, ABC1, ABF2, AC01, ACR1, ADH3, ADK2, AEP2, AFG3, ALD1, ALD2, ARG11, ARG2, ARG5,6, ARG7, ARG8, ARH 1, AT M 1, ATP1, ATP10, ATP11, ATP12, ATP14, ATP15, ATP16, ATP2, ATP3, ATP4, A TP5, ATP6, ATP7, ATP8, ATP9, BAT1, BCS1, CBP1, CBP2, CBP3, CBP4, CB P6, CBR1, CBS1, CBS2, CCA1, CCE1, CCP1, CEM1, CIT1, CIT3, COB, CO Q1, COQ2, COQ3, COQ6, COR1, COT1, COX1, COX10, COX11, COX12, C 0X3, COX14, COX15, COX17, COX2, COX3, COX4, COX5A, COX5B, COX6, COX7, COX8, COX9, CPR3, CTP1, CYB2, CYC1, CYC2, CYC3, CYC7, CYT1, CYT2, DB156, DLD1, DTP, ENS2, ERV1, FLX1, FUM1, GCV1, GCV3, GI-04, GPD2, GSD2, GUT2, HEM1, HEM15, HSP10, HSP60, HSP78, HTS1, IDH1, ID H2, IDP1, IFM1, ILV1, ILV2, ILV3, ILV5, ILV6, IMP1, IMP2, INH1, ISM1, KG D1, KGD2, LAT1, LEU4, LIP5, LPD1, LYS12, LYS4, MAE1, MAM33, MAS1, MAS2, MBA1, MCR1, MDH1, MDJ1, MDJ2, MDM10, MDM12, MEF1, MEF2, MET13, MGE1, MGM101, MIP1, MIR1, MIS1, MMM1, MMTi, M MT2, MOD5, MOL1, MRF1, MRP1, MRP13, MRP17, MRP2, MRP20, MRP21, MRP4, MRP49, MRP51, MRP8, MRPL10, MRPL 11, MRPL13, MRPL15, MRPL16, MRPL 17, MRPL 19, MRPL2, MRPL20, MRPL23, MRPL24, MRPL25, MRPL27, MRPL28, MRP L3, MRPL31, MRPL32, MRPL33, MRPL35, MRPL36, MRPL37, MRPL38, MR PI-39, MRPL4, MRPL40, MRPL44, MRPL49, MRPL6, MRPL7, MRPL8, M RPL9, MRPS28, MRPS5, MRPS9, MRS1, MRS11, MRS2, MRS3, MRS4, MRS5, MSD1, MSE1, MSF1, MSH1, MSK1, MSM1, MSP1, MSR1, MS S1, MSS116, MSS18, MSS51, MST1, MSU1, MSW1, MSY1, MTF1, M T01, NAM1, NAM2, NAM9, ND11, NHX1, NUC1, OM45, ORFA04514, OSM1, OXA1, PDA1, PDB1, PDX1, PEL1, PET111, PET112, PET117, PET122, PET123, PET127, PET130, PET191, PET309, PET494, PET54, PET56, PET9, PETCR46, PI-1131, PHB2, PIF1, PIM1, POR1, POR2, PPA2, PSD1, PUT1, PUT2, QCR10, QCR2, QCR6, QCR7, QCR8, QCR9, RCAi, RF2, RIM 1, RIM2, RIP1, RML2, RNA12, RPM2, RP 041, SC01, SCO2, SDI-11, SDH2, SDI-13, SDI-14, SECY, SHM1, SHY1, SLS1, SMF2, SOD2, SOM1, SSC1, SS.COPYRGT.1, STF1, STF2, SUN4, SUV3, TIM17, TIM22, TIM23 TIM44, TIM54, TOM20, TOM22, TOM37, TOM40, TOM6, TOM7, TOM 70, TOM72, TRM1, TUF1, UNG1, VAR1, YAH1, YAL011W, YAT1, YBL013 W, YCR024C, YDR041W, YDR115W, YDR116C, YER073W, YFH1, YGLO68W, YGR257C, YHM1, YHR075C, YHR148W, YJL200C, YJR113C, YKLO55C, YKL120W, YKL134C, YKL192C, YLR168C, YMC1, YMC2, YML025C, YMR188C, YMR31, YNL081 C, YNL306W, YNR036C, YNR037C, YOR221 C, YPL013C, ETF-BETA

PEROXISOMAL CAT2, CIT2, CTA1, DAL7, EHD1, EHD2, FAA2, FAT2, FOX2, ICU, IDP 3, MDH3, MLS1, PEX11, PEX12, PEX13, PEX14, PEX17, PEX2, PEX3, PE X4, PEX6, PEX7, PEXB, POT1, POX1, PXA1, PXA2, SPS19, YBR204C, YDR 449C, YHR180W DNA-ASSOCIATED A1, A2, ABF1, ABF2, ADA2, ADE12, ADR1, ALPHA1, ALPHA2, ANC1, APN1, ARGR1, ARGR2, ARGR3, ARR1, ASH1, AZF1, BAS 1, BDF1, BR F1, BUR6, CAC2, CAD1, CAF17, CATB, CBF1, CBF2, CCE1, CCR4, CDC13, CDC36, CDC39, CDC46, CDC47, CDC54, CDC6, CDC7, CDC73, CDC9, CEF1, CEP3, CHA4, CHD1, CHU, CHL4, CRZ1, CSE1, CSE2, CSE4, CTF13, CUP2, CUP9, DAL80, DAL81, DAL82, DAT1, DBF4, DMC1, DNA2, DNA43, DNL4, D OS2, DOT6, DP131 1, DPB2, DPB3, DST1, ECM22, ENS2, EST1, EZL1, FCP1, FHL1, FKH1, FKH2, FL08, FZF1, GAL11, GAL4, GAT1, GBP2, GCN4, GCNS, GCR1, GCR2, GLN3, GL03, GTS1, GZF3, HAC1, HAP1, HAP2, HAP3, HAP4, HCM1, HDA1, HDF1, HFM1, HHF1, HHF2, HH01, HHT1, HHT2, HM01, HMS1, HMS2, H0, HOP1, HPR1, HPRS, HSF1, HTA1, HTA2, HTA3, HTB1, HT 62, IFH1, IME1, IME4, IN02, IN04, IXR1, KAR4, LEU3, LYS14, LYS20, LYS21, M AC 1, MAGI, MAL13, MAL23, MAL33, MATALPHA1, MATALPHA2, MBP1, MCD1, MCM1, MCM2, MCM3, MCM6, MED6, MER2, MET18, MET28, MET30, MET31, MET32, MET4, MGA2, MGT1, MIF2, MIG1, MIG2, MIP1, MLH 1, MOL1, MOT1, MPT4, MRE11, MSH1, MSH2, MSH3, MSH4, MSHS, MS11, MSN1, MSN2, MSN4, MTF1, NBN1, NC132, NDJ1, NGG1, NHP2, NHP6 A, NHP6B, NOT3, NUC2, OAF1, OP11, ORC1, ORC2, ORC3, ORC4, ORCS, ORC6, PAF1, PCH1, PCH2, PDR1, PDR3, PGD1, PHD1, PH02, PH04, PHR1, PIF1, PIP2, PMS1, POB1, POL1, POL12, POL2, POL3, POL30, PO L4, POP2, PPR1, PRI1, PR12, PS02, PUT3, RAD1, RAD10, RAD14, RAD 16, RAD18, RAD2, RAD23, RAD26, RAD27, RAD3, RAD4, RADS, RAD50, RAD51, RAD52, RAD54 RAD55, RAD57, RAD6, RAD7, RAP1, RAT-1, RCS1, REB1, REC102, RE C104, REC114, RED1, REG1, RET1, REV3, RFA1, RFA2, RFA3, RFC1, RFC2, RFC3, RFC4, RFCS, RGM1, RGT1, RIF1, RIF2, RIM1, RIM101, RLF2, RLM1, R ME1, RMS1, ROX1, ROX3, RPA12, RPA135, RPA14, RPA190, RPA34, RPA43, RPA49, RP131 0, RPB11, RPB2, RP133, RP134, RPBS, RPB6, RPB7, RPBB, RPB9, RPC10, RPC19, RPC25, RPC31, RPC34, RPC40, RPC53, RPC82, RPD3, RP021, RP031, RP041, RRN10, RRN11, RRN3, RRNS, RRN6, RRN7, RRN9, RSC4, RSC 6, RSC8, RTG1, RTG3, SASS, SEF1, SET1, SFH1, SFL1, SGS1, SIG1, SIN 3, SIN4, SIP2, SIP4, SIR1, SIR2, SIR3, SIR4, SKN7, SK01, SMC1, SMC 2, SMP1, SNF2, SNFS, SNF6, SOK2, SPKi, SPOL, SPS18, SPT10, SPT 15, SPT16, SPT2, SPT21, SPT23, SPT3, SPT4, SPT5, SPT6, SPTB, SRI 32, SRB4, SRBS, S RB6, SRB7, SR138, SR139, SSL2, SSN3, SSN6, SSNB, SSU72, STB4, STBS, S TE12, STH1, SUA7, SWI1, SW13, SW14, SW16, SWP73, TAF19, TAF25, TBF1, TEA1, TEC1, TFAI, TFA2, TF131, TF132, TF133, TFB4, TFC1, TFC2, TF C3, TFC4, TFCS, TFG1, TFG2, TH12, TOA1, TOA2, TOP1, TOP2, TOP3, TRF4, TS P1, TUP1, TYE7, UGA3, UME6, UNGI, USV1, XRS2, YAL019W, YAP1, YA P3, YAPS, YBL054W, YBR026C, YBR150C, YBR239C, YCR106W, YDR026 C, YDR060W, YDR213W, YER045C, YER184C, YFL052W, YIL036W, YIL 130W, YJL103C, YJL206C, YKL005C, YKL222C, YKR064W, YLL054C, YLRO 87C, YLR266C, YNL206C, YOL089C, YOR172W, YOR380W, YOX1, YPL133C, YPR008W, YPR196W, YRR1, ZAP1, ZIP1, ZU01

IMMUNOSUPPRESSANT FEN1, SSH4, SHR3 CYCLINS CCU, CLB1, CLB2, CLB3, CL134, CLBS, CLB6, CLG1, CLN1, CLN2, CLN 3, CTK2, PCL1, PCL10, PCL2, PCLS, PCL6, PCL7, PCLB, PCL9, PH080, S SNB, YBR095C

ATP-BINDING CASSETTE PROTEINS ADP1, ATM 1, CAF16, GCN20, MDL1, MDL2, PDR10, PDR11, PDR12, PDR15, PDRS, PXA1, PXA2, SNQ2, STE6, YBT1, YCF1, YDL223C, YD R091C, YEF3B, YER036C, YHL035C, YKR103W, YKR104W, YLL015W, YNR070W, YOR011W, YOR1, YPL226W

CYTOSKELETAL ABP1, ACF2, ACT1, AFR1, AIP1, AIP2, ARP3, AUT2, AUT7, BEM1, BI M1, BN11, BN14, BUD3, BUD6, CAP1, CAP2, CDC10, CDC11, CDC12, CDC 3, CIN1, CIN2, CIN4, CMD1, COF1, CRN1, END3, GIC1, GIC2, GIN4, J NM1, KAR9, KIP2, KIP3, LAS 17, MDM1, MHP1, MYO01, MY02, MY03, MY04, MY05, PFY1, RVS161, RVS167, SAC6, SAC7, SEC 1, SHE3, SHM2, SLA1, SL A2, SMY1, SMY2, SPA2, SPH1, SPR28, SPR3, SRV2, TCP1, TPM1, TPM2, TUB1, TUB2, TUB3, VPS16, VRP1 APOPTOSIS ATP1, ATP14, ATP15, ATP16, ATP2, ATP3, ATP4, ATPS, ATP6, ATP7, ATP8, ATP9, CYC1, SH01, SSK2, SSK22, SW13, SXM1

CELL RESCUE ACC1, ALD6, BCK1, BEM 1, BEM2, BIM1, BMH1, BMH2, CAN1, CBF1, CDC1, CDC14, CDC15, CDC20, CDC25, CDC28, CDC33, CDC37, CDC 42, CDC43, CDC53, CDC6, CHC1, CIN8, CKA1, CKA2, CLA4, CLB1, CLB2, CLB3, CLB4, CLB5, CLN1, CLN2, CLN3, CMP2, CNA1, COF1, CTT1, DBF2, DBF20, DPM1, ERG25, GIC1, GIC2, GPA1, GRR1, HCA4, HIS4, HOC1, HSF1, KAR1, KES1, KRE6, KSS1, MBP1, NMT1, ORC2, ORC5, PDE2, PEP12, PEP7, PKC1, P LC1, PMR1, POL30, PRP18, RAM1, RAS1, RAS2, RBL2, RED1, RFC1, RH01, RH03, RHO4, SAC1, SEC13, SEC14, SEC22, SEC4, SET1, SIS2, SKP1, SPC98, SRA1, SR04, SRP1, SSA1, SSA2, SSA4, SSN8, STE20, STN 1, STT4, SUJ 3, SWE1, SW14, SW16, TEL1, TOR1, TUB1, TUB4, VMA1, YCK1, YCK2, YPT1

CELL DAMAGE APN1, BUB1, CDC28, CDC45, CDC46, CDC47, CDC54, CDC7, CLB1, CLB2, CLB3, CLB5, DDC1, DDR2, DDR48, DIN7, DUN1, ECM32, HSM3, IMP2, MEC1, MEC3, MGT1, MOL1, MRE11, MUS81, NTG1, PDS1, PGD1, P HR1, POL2, POL3, POL30, POL4, PR11, PS02, RAD14, RAD16, RAD17, RAD18, RA D24, RAD30, RAD51, RAD52, RAD54, RAD55, RAD57, RAD7, RAD9, RDH54, REV3, RFA1, RFC5, RNR1, RNR2, RNR3, RNR4, RPH1, SIC1, SML1, SP K1, STN1, STS1, TEL1, TFA1, TFA2, TUP1, UBC7, UB14, XBP1, YBR098W, YFH1

OTHER RELEVANT MUTANTS AND GENES Y-1,9520b, C658-K7, JPD 4, JPM 9, Cy32, E354, JC488, PSY 142, 01-2, Y217, JC787-9A, ML1-21, Y500,86-9C, GL1, GT5-1A, HD565A, PZ1, 127-4D, Y229, JC302-26B, JC482, LB2211-2B, MH41-7B/P21, erg 81, SEY6211, GL4, K335, MK20, MK34, DE4-3A, DE4-3B, DE4-3C, MMY011, UH 1-GRGZ, 2150-2-3a, Y211, DP1/517,943,1117, C658, 1252, H79.20.3, LB1-3B, C658-K42, R29B, LB54-3A, XW520-9A, ade7, D225-5A, 309, SDH1, SDH2, SDH3, SDH4, TCM62, PDE1, PDE2

Mammalian Genes:

11-beta hydroxysteroid dehydrogenase type II, 12-lipoxygenase, 17-beta hydroxysteroid dehydrogenase, 60S ribosomal protein L6,6-Omethylguanine-DNA methyltransferase, Activating transcription factor 2, Activating transcription factor 3, Activating transcription factor 4, Activin beta E, Activin receptor type 11, Acyl-CoA dehydrogenase, Acyl CoA Carrier Protein, Adenine nucleotide translocator 1, Alanine aminotransferase, Alcohol dehydrogenase 1, Alcohol dehydrogenase 2, Alcohol dehydrogenase 3, Alcohol dehydrogenase 4, Alcohol dehydrogenase 5, Aldehyde dehydrogenase 1, Aldehyde dehydrogenase 2, Aldehyde dehydrogenase 3, Alpha 1-antitrypsin, Alpha-1 acid glycoprotein, Alpha-1 antichymotrypsin, Alpha-catenin, Alphatubulin, Apolipoprotein A1, Apolipoprotein A11, Apolipoprotein Clil, Apolipoprotein E, Aryl hydrocarbon receptor, Aspartate aminotransferase, mitochondrial, Ataxia telangeictasia, ATP-dependent helicase 11 (70 kDa), ATP-dependent helicase 11 (Ku80), BAG-1, BAK, Bax (alpha), Bcl-2, Bcl-xL, Beta-actin, Bilirubin UDP-glucuronosyl-transferase isozyme 1, Bilirubin UDP-glucuronosyl-transferase isozyme 2, Biliverdin reductase, Branched chain acylCoA oxidase, BRCA1, BR-cadherin, C4bbinding protein, c-abl, Calcineurin B, Calnexin, Calprotectin, Calreticulin, canalicular multispecific organic anion transporter, Carbonic Anhydrase 111, Carnitine palmitoyl-CoA transferase, Caspase 1, Caspase 2 (Nedd2), Caspase 3 (CPP32beta), Caspase 5 (ICE relIII), Caspase 6 (Mch2-alpha), Caspase 7 (Mch3alpha), Caspase 8 (FLICE), Catalase, CatecholOmethyltransferase, CCAAT/enhancer-binding protein alpha, CCAAT/enhancer-binding protein epsilon, Cell division cycle protein 2, Cell division cycle protein 20, Cell division cycle protein 25, Cellular retinoic acid binding protein 1, Cellular retinoic acid binding protein 2, cerb; c-fos, Checkpoint kinase-1, Cholesterol esterase, c-H-ras, cjun, Clusterin, c-myc, Complement component C3, Connexin 30, Connexin32, Connexin-40, Corticosteroid binding globulin, Corticotropin releasing factor, C-reactive protein, Creatine kinase b, Cyclin D1, Cyclin dependent kinase 1, Cyclin dependent kinase 4, Cyclin dependent kinase inhibitor 1A, Cyclin E, Cyclin G, Cyclin-dependent kinase 4 inhibitor (P116), Cyclindependent kinase 4 inhibitor B (P16), Cyclin-dependent kinase inhibitor P27Kip1, Cyclooxygenase 2, Cystic fibrosis transmembrane conductance regulator, Cytochrome P450 11A1, Cytochrome P450 17A, Cytochrome P450 1A1, Cytochrome P450 1A2, Cytochrome P450 1B1, Cytochrome P450 2A 1, Cytochrome P450 2A3, Cytochrome P450 2A6, Cytochrome P450 2131, Cytochrome P450 21310, Cytochrome P450 2132, Cytochrome P450 2C11, Cytochrome P450 2C12, Cytochrome P450 2C19, Cytochrome P450 2C9, Cytochrome P450 2D6, Cytochrome P450 2E1, Cytochrome P450 2F2, Cytochrome P450 3A1, Cytochrome P450 3A4, Cytochrome P450 4A, Cytochrome P450 4A1, Damage specific DNA binding protein p48 subunit, Defender against cell death-1, Deleted in colorectal cancer, Deltalike protein, Dihydrofolate reductase, Disulfide isomerase related protein (ERp72), DNA binding protein inhibitor ID2, DNA dependent helicase, DNA dependent protein kinase, DNA ligase 1, DNA ligase IV, DNA mismatch repair protein (MLH1), DNA mismatch repair protein (PMS2), DNA mismatch repair/binding protein (MSH3), DNA polymerase alpha, DNA polymerase beta, DNA polymerase beta, DNA repair and recombination homologue (RAD 52), DNA repair helicase II ERCC-3, DNA repair protein (RAD 50), DNA repair protein (XRCC1), DNA repair protein XP-D, DNA replication factor C (36 kDa), DNA topoisomerase 1, DNA topoisomerase 11, Dopamine beta-hydroxylase, DRA, Dynein light chain 1, E2F, Early growth regulated protein 1, E-Cadherin, ECE-1 (endothelin converting enzyme), Endothelin-1, Enolase alpha, Enoyl CoA hydratase, Eotaxin, Epidermal growth factor, Epoxide hydrolase, ERA-B, ERCC 1 (excision repair protein), ERCC 3 (DNA repair helicase 11), ERCC 5 (excision repair protein), ERCC 6 (excision repair protein), ERK1, Erythropoietin, Erythropoietin receptor, ESelectin, Estrogen receptor, Farnesol receptor, Fas antigen, Fas associated death domain (FADD), Fas ligand, Fas/Apo1 receptor, Fatty acid synthase, Fatty acyl-CoA oxidase, Fatty acyl-CoA synthase, FEN-1 (endonuclease), Fibrinogen gamma chain, Fibronectin receptor, FIC1, Filagrin, Flavin containing monooxygenase 1, Flavin containing monooxygenase 3, FosB, Fra-1, Fucosyl transferase (alpha-1,2fucosyltransferase), Gadd153, Gadd45, Gamma-glutamyl hydrolase precursor, Gamma-glutamyl transpeptidase, GCLR, GCLS, Glucocorticoid receptor, Glucose-6-phosphate dehydrogenase, Glucose-regulated protein 170, Glucose-regulated protein 58, Glucose-regulated protein 78, Glucoseregulated protein 94, Glutamicoxaloacetic transaminase, Glutaminc-pyruvic transaminase, Glutathione peroxidase, Glutathione reductase, Glutathione S-transferase alpha subunit, Glutathione S-transferase 4a, Glutathione synthetase, Glyceraldehyde 3-phosphate dehydrogenase, GOS24 (zinc finger transcriptional regulator), Granulocyte-macrophage colony-stimulating factor, Growth-arrested-specific protein 1, Growth-arrested-specific protein 3, GT mismatch binding protein, H-cadherin, Heat shock protein 12, Heat shock protein 47, Heat shock protein 70, Heat shock protein 70.1, Heat shock protein 90, Helicase-like transcription factor, Heme binding protein 23, Heme oxygenase-1, Hepatic lipase, Hepatocyte growth factor, Hepatocyte growth factor activator, Hepatocyte growth factor receptor, Hepatocyte nuclear factor 4, Histone 2A, Histone 28, HMG CoA reductase, Hydroxyacyl CoA dehydrogenase, Hydroxysteroid sulfotransferase a, Hypoxanthine-guanine phosphoribosyltransferase, ICE-rel 11 (Caspase 4), ICH-2 cysteine protease=CASPASE 4, IkB-a, Insulin-like growth factor binding protein 1, Insulin-like growth factor binding protein 2, Insulin-like growth factor binding protien 3, Insulin-like growth factor I, Insulin-like growth factor 11, Integrin alpha, Integrin alpha L, Integrin betas, Integrin beta2, Intercellular adhesion molecule-1, Intercellular adhesion molecule-2, Intercellular adhesion molecule-3, Interferon gamma, Interferon inducible protein 10, Interferon inducible protein 15, Interleukin-1 alpha, Interleukin-12, Interleukin-2, Interleukin-4, Interleukin-5, Interleukin-6, Involucrin, JNK1 stress activated protein kinase, K-cadherin, Ki67, Lactate Dehydrogenase 8, Lactoferrin, Lipopolysaccharide binding protein, Lipoprotein lipase precursor, Liver fatty acid binding protein, L-myc, Low density lipoprotein receptor, Luteinizing hormone, Lysyl oxidase, Macrophage inflammatory protein-1 alpha, Macrophage inflammatory protein-1 beta, Macrophage inflammatory protein-2 alpha, Macrophage inflammatory protein-2 beta, Macrophage inflammatory protein-3 alpha, Macrophage inflammatory protein-3 beta, Malic enzyme, MAP kinase kinase, Matrix metal loproteinase1, Matrix metal loproteinase-2, MDM-2, MET proto-oncogene, Metallothionein 1, Metallothionein 2, Metallothionein 3, Metallothionein IA, Metallothionein IG, Metalregulatory transcription factor-1, Mitogen activated protein kinase (P38), Mitogen inducible gene (mig-2), MOAT-B (MRP/organic anion transporter), Monoamine oxidase A, Monoamine oxidase B, Multidrug resistance-associated protein, Multidrug resistant protein- 1, Multidrug resistant protein-2, Multidrug resistant protein-3=cMOAT2, MUTL homologue (MLH1), MutS Homologue (MSH2), Myeloid cell differentiation protein-1, Na/taurocholate cotransporting polypeptide, NADPH cytochrome P450 -oxidoreductase, NADPH cytochrome P450 reductase, NADPH quinone oxidoreductase-1 (DTDiaphorase), Natural killer cell-enhancing factor B, N-cadherin, NF-kappaB (p65), Nitric oxide synthase-1, inducible, Nucleoside diphosphate kinase beta isoform, 0-6-alkylguanine-DNAalkyltransferase, OBcadherin 1, OB-cadherin 2, Octamer binding protein 1, Octamer binding protein 2, Octamer binding protein 3, Oncostatin M, Organic anion transporter 1, Organic anion transporter 3, Organic anion transporter K1, Organic anion transporting polypeptide 1, Organic cation transporter 1, Organic cation transporter 2, Organic cation transporter 3, Organic cation transporter N1, Organic cation transporter N2, Ornithine decarboxylase, Osteopontin, Oxygen regulated protein 150, p53, PAPS synthetase, P-cadherin, PEGS (progression elevated gene 3), Peroxisomal 3-ketoacyl-CoA thiolase 1, Peroxisomal 3-ketoacylCoA thiolase 2, Peroxisomal acyl-CoA oxidase, Peroxisomal fatty acyl-CoA oxidase, Peroxisome assembly factor 1, Peroxisome assembly factor 2, Peroxisome biogenesis disorder protein-1, Peroxisome biogenesis disorder protein-11, Peroxisome biogenesis disorder protein-4, Peroxisome hydratase, Peroxisome proliferator activated receptor alpha, Peroxisome proliferator activated receptor gamma, Phenol sulfotransferase, Phosphoenolpyruvate carboxykinase, Phosphoglyceride kinase, Phospholipase A2, Plasminogen activator inhibitor 2, Platelet derived growth factor B, Platelet/endothelial cell adhesion molecule-1, Poly (ADP ribose) polymerase, Proliferating cell nuclear antigen gene, Prostaglandin H synthase, Protein kinase C betal, Protein-tyrosine phosphatase, Putative protein tyrosine phosphatase, RAID, RAID 51 homologue, RANTES, Ref 1, Replication factor C, 40-kDa subunit (Al), Replication protein A (70 kDa subunit), Retinoblastoma, Retinoblastoma related protein (P 107), Retinoid X receptor alpha, Retinoid X receptor beta, Retinoid X receptor gamma, Ribonucleotide reductase M1 subunit, Ribosomal protein L13A, Ribosomal protein S9, RNA-dependent helicase, ROAT1 (renal organic anion transporter), Serum amyloid A1, Serum amyloid A2alpha, Sister of p-glycoprotein, Sodium/bile acid cotransporter, Sonic hedgehog gene, SQM1, Superoxide Dismutase Cu/Zn, Superoxide dismutase Mn, T-cell cyclophilin, Tenascin, Thiopurine methyltransferase, Thioredoxin, Thrombospondin 2, Thymidine kinase, Thymidylate synthase, Thymosin beta-10, Tissue inhibitor of metalloproteinases-1, Tissue transglutaminase, Transcription factor IID, Transferrin, Transforming growth factor-beta 3, Tumor necrosis factor associated factor 2 (TRAF2), Tumor necrosis factor receptor 1, Tumor necrosis factor receptor 2, Tumor necrosis factor receptor-1 associated protein (TRADD), Tumor necrosis factor-alpha, Tumor necrosis factorbeta, Type 1 interstitial collagenase, Tyrosine aminotransferase, Tyrosine protein kinase receptor (UFO), Ubiquitin, Ubiquitin conjugating enzyme (Rad 6 homologue), Ubiquitin-homology domain protein PIC1, UDPglucuronosyltransferase 1, UDP-glucuronosyltransferase 1A6, UDPglucuronosyltransferase 2, UDP-glucuronosyltransferase 28, Uncoupling protein 1, Uncoupling protein 2, Uncoupling protein 3, Urate oxidase, UV excision repair protein RAD 23 (XP-C), Vascular cell adhesion molecule 1 (VCAM-1), Vascular endothelial growth factor, Vascular endothelial growth factor D, Very long-chain acyl-CoA dehydrogenase, Vimentin, Vitellogenin, Waf1, XRCC1 (DNA repair protein).

Passenger genes also include genes encoding prodrug-activating enzymes such as viral thymidine kinase, viral thymidine phosphorylase, cytosine deaminase, bacterial carboxypeptidase G2, cytochrome P450 proteins, carboxylesterase, deoxycytidine kinase, nitroreductase, purine nucleoside phosphorylase, horseradish peroxidase, and xantine guanine phosphoribosyl transferase. Also included are genes encoding “proteotoxins” such as diphtheria toxin and cytolethal distending toxin.

The present invention, thus generally described, will be understood more readily by reference to the following examples, which are provided by way of illustration and are not intended to be limiting of the present invention.

### EXAMPLES

Construction of an Adenovirus Pair that Conditionally Replicates in Infected Cells Exposed to Small Molecule Regulator Mifepristone and Transient Heat

Adenovirus type 5 is abbreviated Ad below. Generally known molecular biology and biochemistry methods are used. Molecular biology methods are described, e.g., in “Current protocols in molecular biology, Ausubel, F. M. et al., eds., volumes 1-4, John Wiley and Sons, Inc. ISBN 0-471-50338-X.

Recombinant Adenovirus 1 (rAd1)

Recombinant adenovirus 1 lacks nucleotides 28,130-30,820 encompassing E3. Nucleotide numbers relating to the adenovirus type 5 genome are as defined in GI: 33694637. Davison, A. J., Benko, M., and Harrach, B. 2003. J. Gen. Virol. 84, 2895-2908. Further, E1A promoter and E4 promoter sequences are replaced with a GAL4 site-containing minimal promoter.

A simplified system for generating recombinant adenovirus developed by He et al. (Proc. Natl. Acad. Sci. USA 95, 2509-2514 (1998)) is employed. A version of this system has bee made available commercially by Stratagene Corp. of La Jolla, Calif. A manual entitled “AdEasy™ Adenoviral Vector System” (revision no. 060002) is distributed by the Company to its customers and is also available at the Company's website www.stratagene.com.

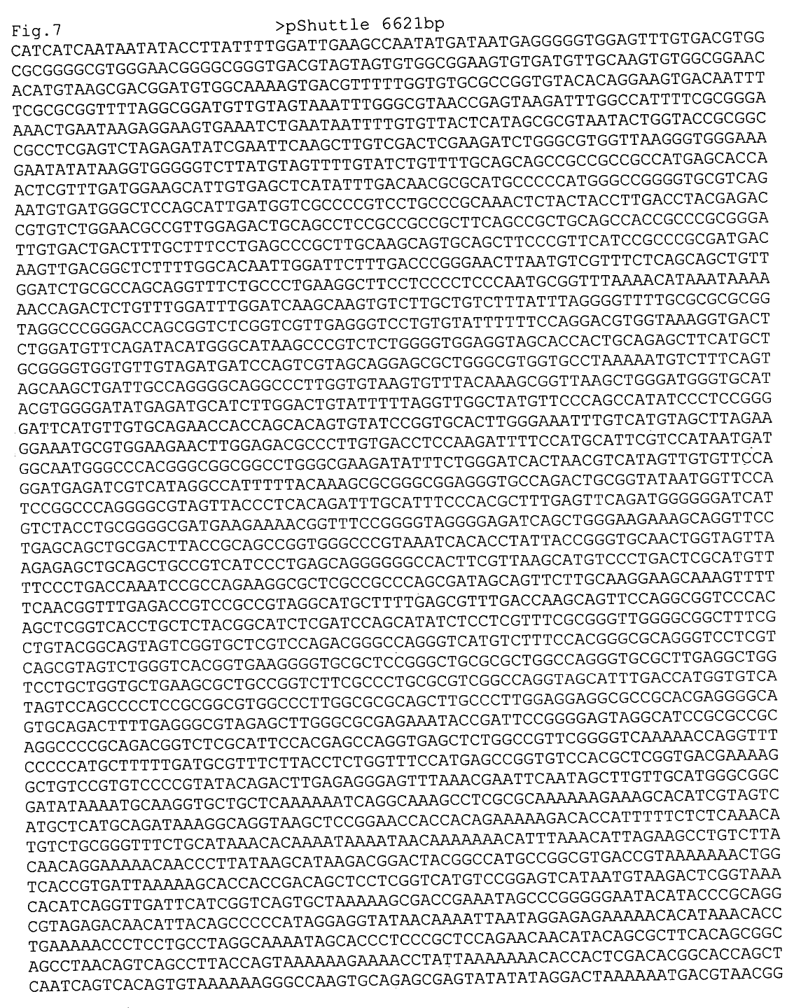

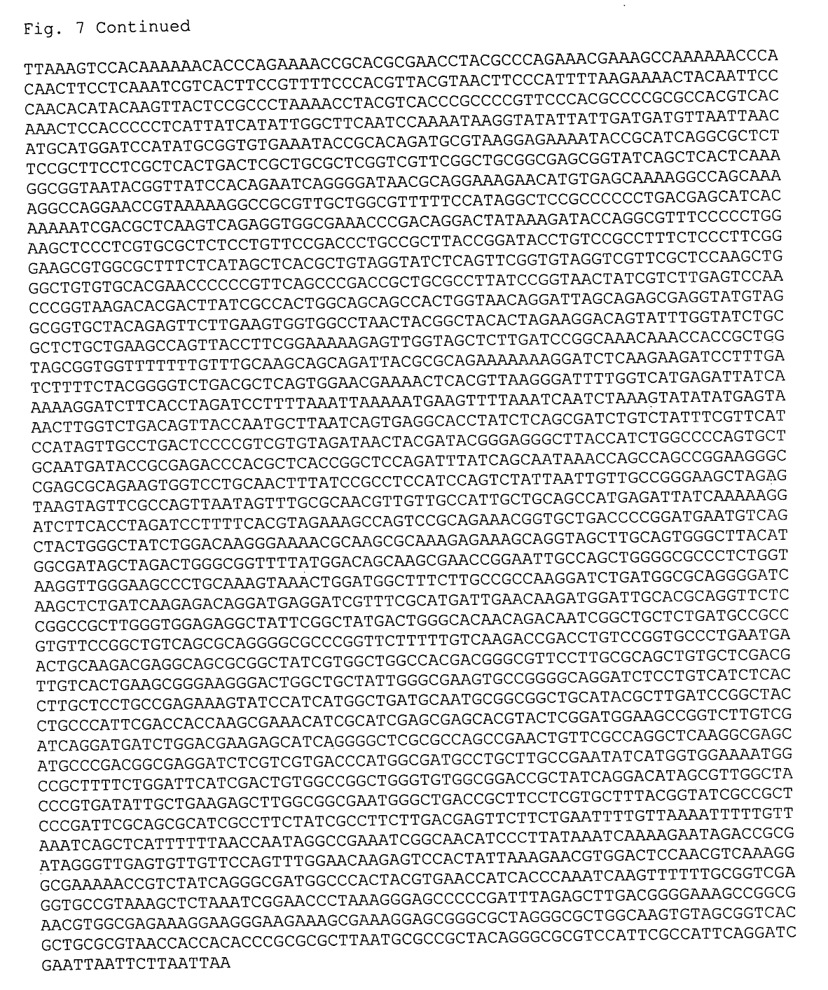

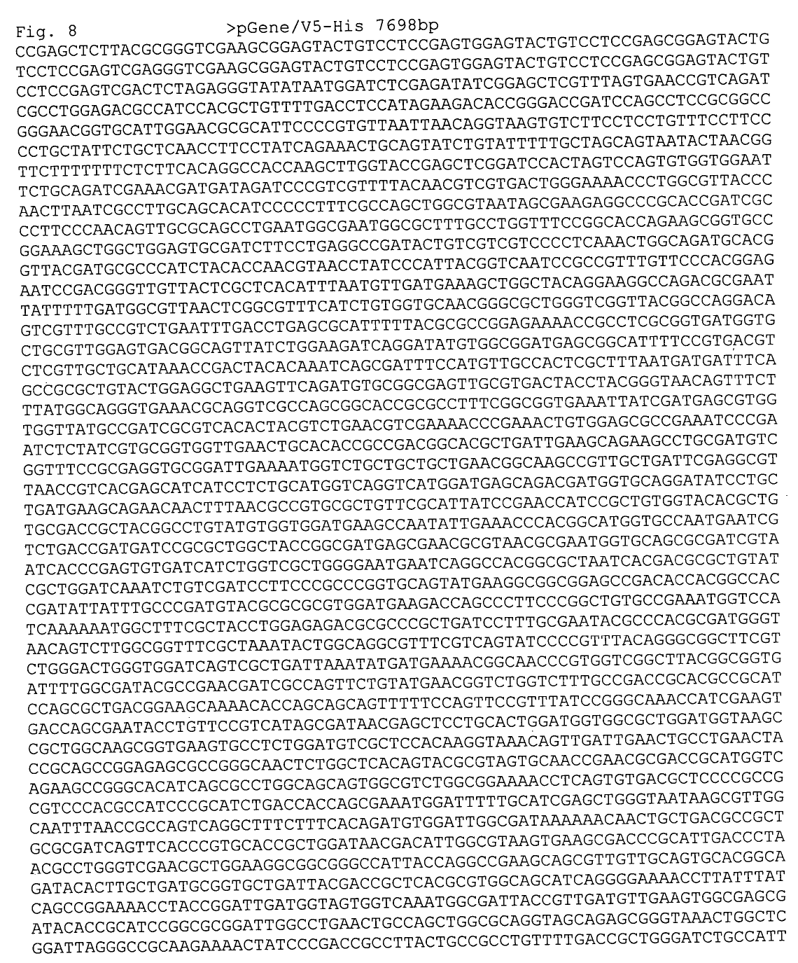

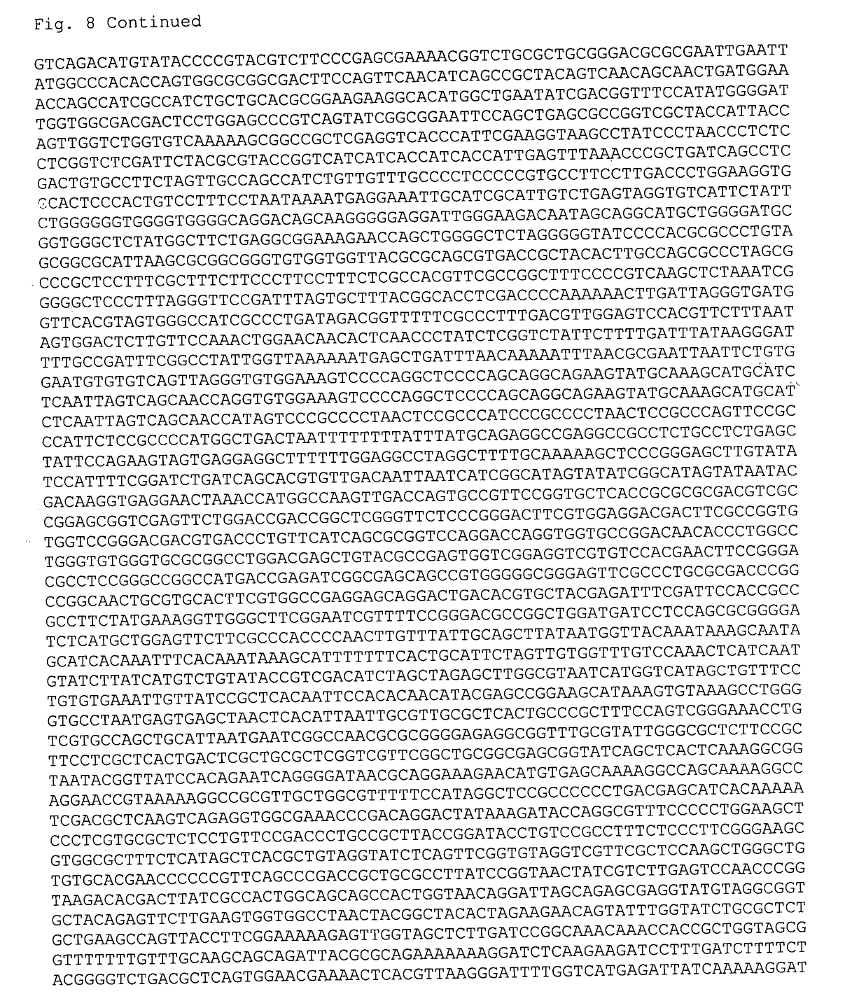

Mutagenesis is carried out in transfer vector pShuttle (He et al. 1998. Proc. Natl. Acad. Sci. USA 95, 2509-2514) distributed by Statagene Corporation. FIG. 7 presents the complete nucleotide sequence of this plasmid. According to Stratagene's manual entitled “AdEasy™ Adenoviral Vector System” (revision no. 060002), pShuttle contains the following adenovirus sequence elements: left inverted terminal repeat, encapsidation signal (Ad 1-331), “right arm homology region” (3,534-5790), “left arm homology region” (34,931-35,935) and right inverted terminal repeat. A site-directed mutagenesis procedure, the so-called QuikChang^{R} procedure of Stratagene Corp. that makes use of the polymerase chain reaction (PCR) is employed to delete E4 promoter sequences present in the left arm homology region of pShuttle (Ad 35,586-35,808) and replace them by an NheI restriction site. The QuikChange protocol is described, for example, in instruction manual “QuikChange XL site-directed mutagenesis kit” (revision no. 063003) published by Stratagene Corp. at their website www.stratagene.com and distributed to customers by mail. A related procedure is described briefly in Guettouche, T. (2002). Ph.D. Thesis, University of Miami, Miami, Fla. The resulting construct is labeled “pShuttle-Nhe”.

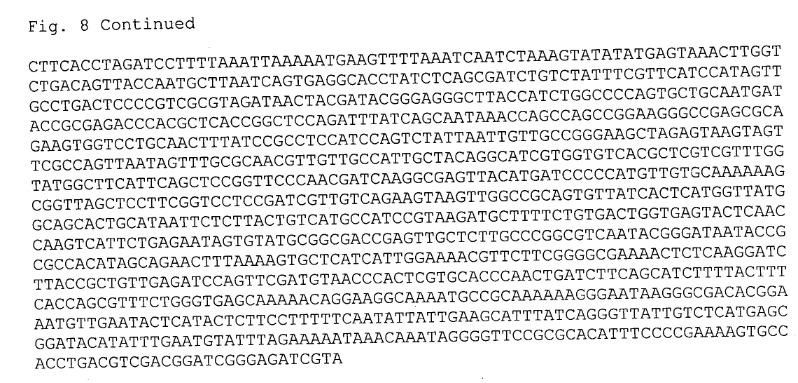

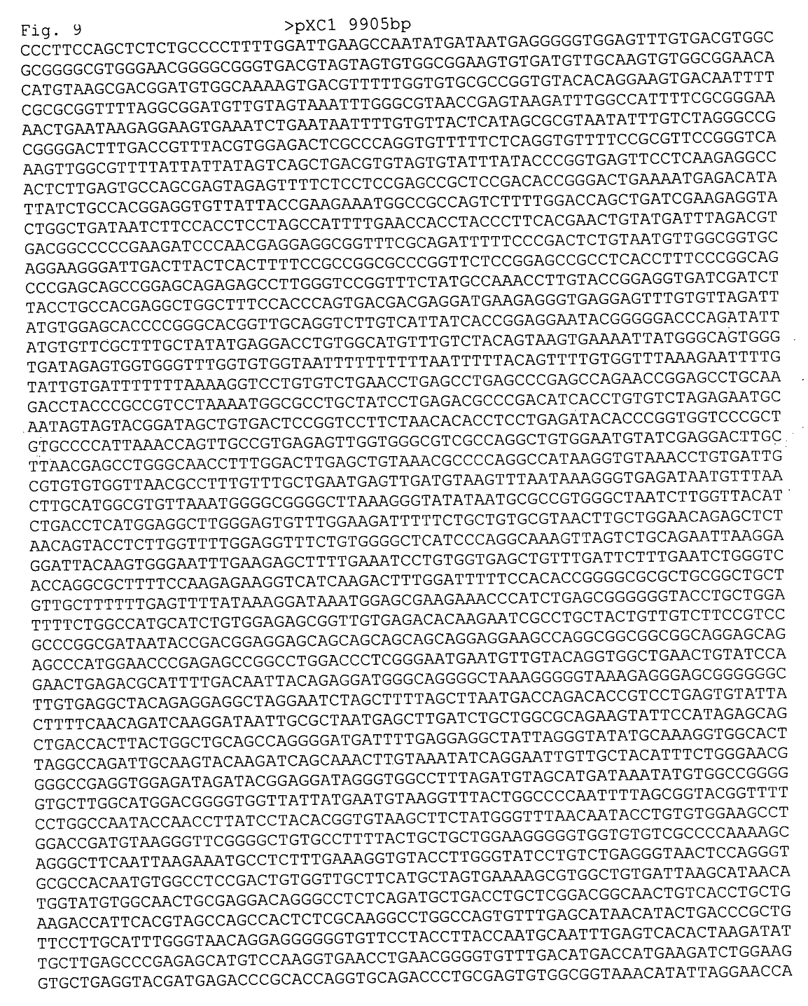

A GAL4-binding site-containing minimal promoter is inserted into the NheI site of pShuttle-Nhe. To achieve this, a DNA fragment encompassing nucleotides 7677 to 323 of plasmid pGene/V5-His (Invitrogen Corporation of Carlsbad, Calif.) is PCR-amplified using appropriate primers also containing an NheI restriction site. After digestion of the PCR fragment with NheI, it is ligated to NheI-digested pShuttle-Nhe DNA. Recombinants are identified by restriction digestion and by nucleotide sequencing. Nucleotide sequencing permits the selection of a clone in which the inserted promoter sequence is correctly oriented relative to the E4 sequences in the left arm homology region of pShuttle-Nhe. This plasmid is named pShuttle-Nhe-GAL. The nucleotide sequence of pGene/V5-His is provided in FIG. 8. The plasmid is also described in instruction manual “GeneSwitch TM System, Version B, 000829/25-0313” of Invitrogen Corp. that is distributed by the Company to its customers and is also available at www.invitrogen.com.

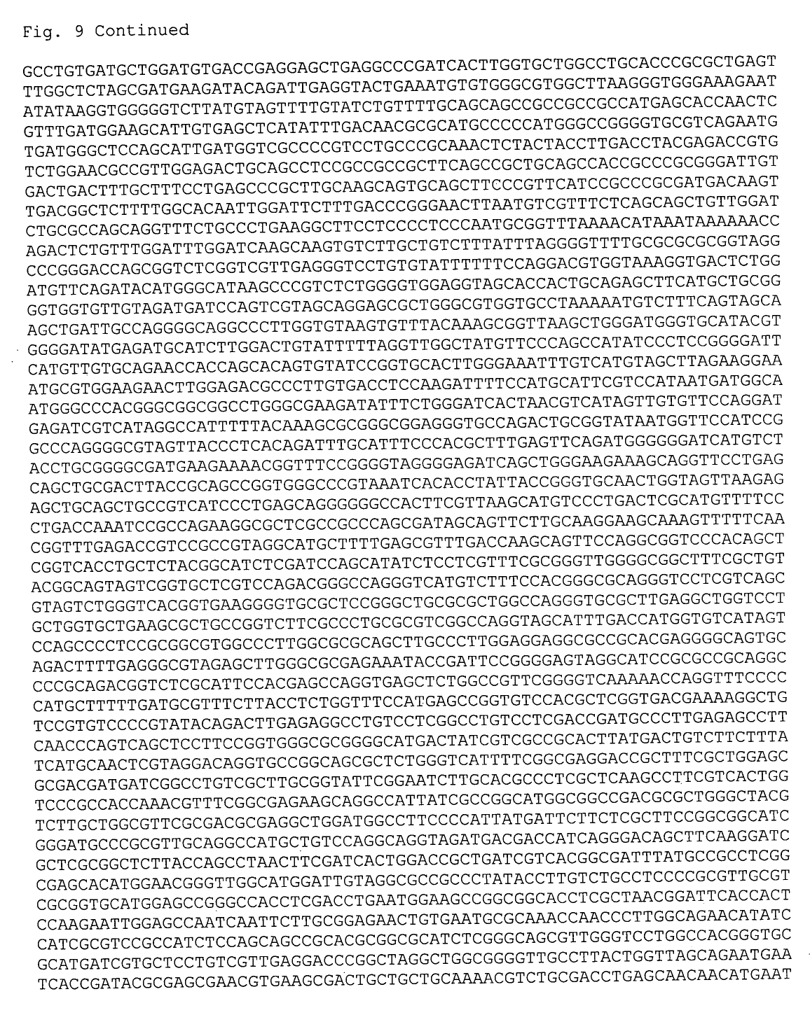

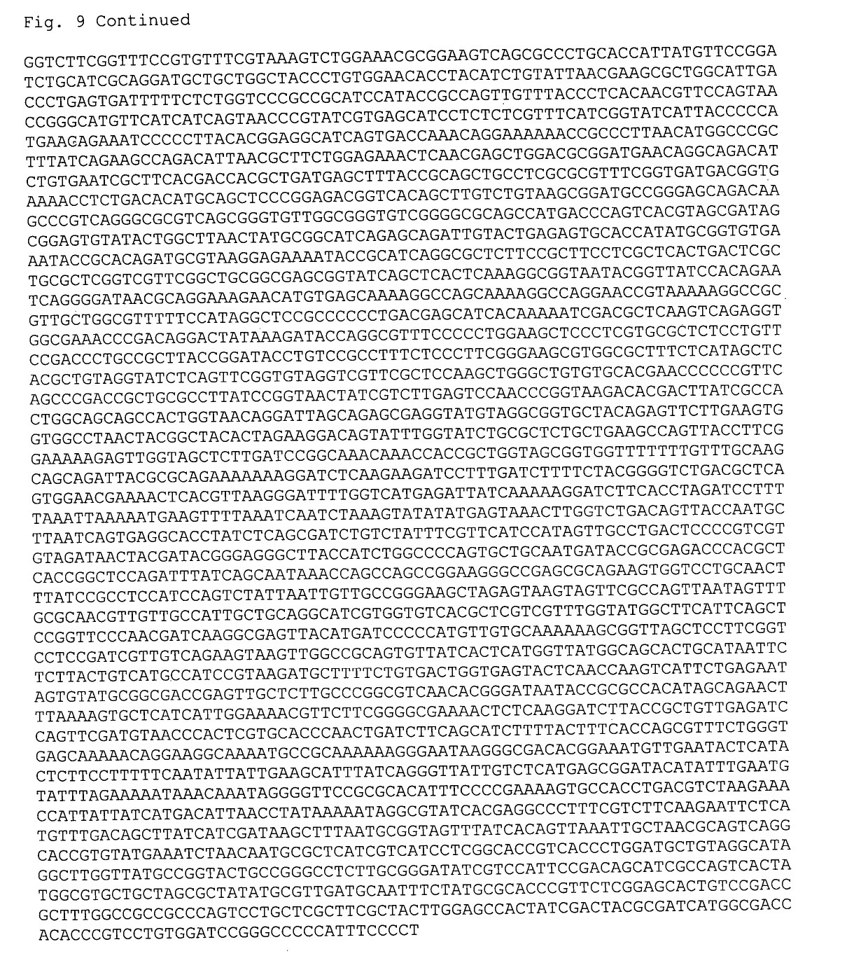

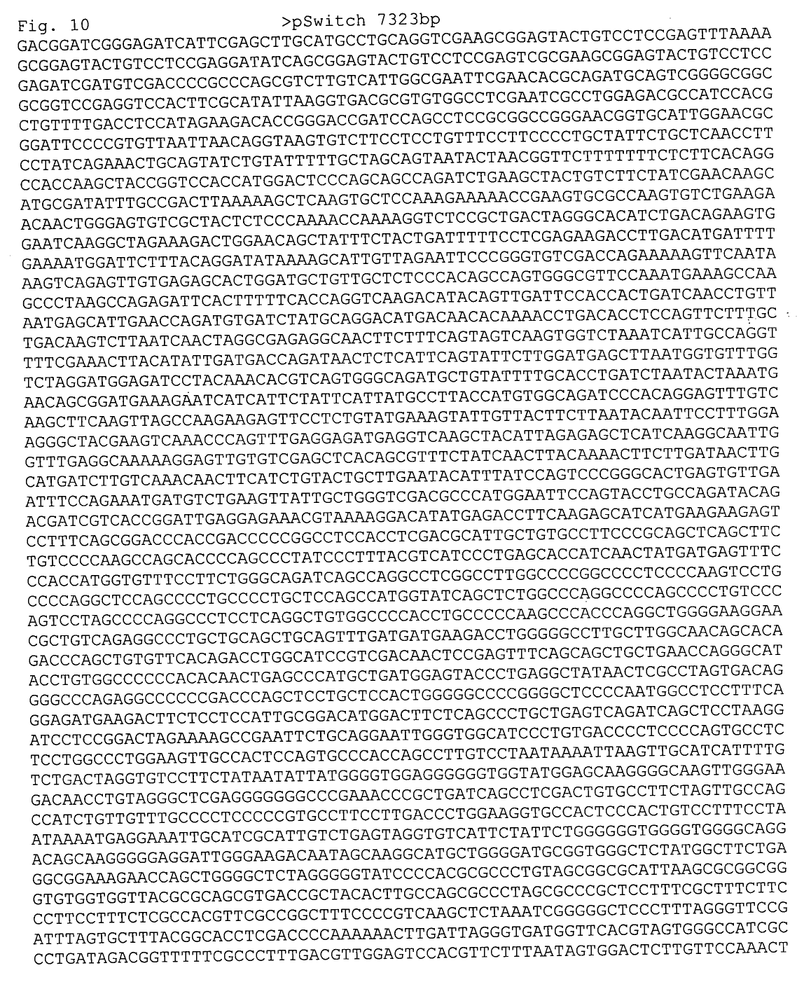

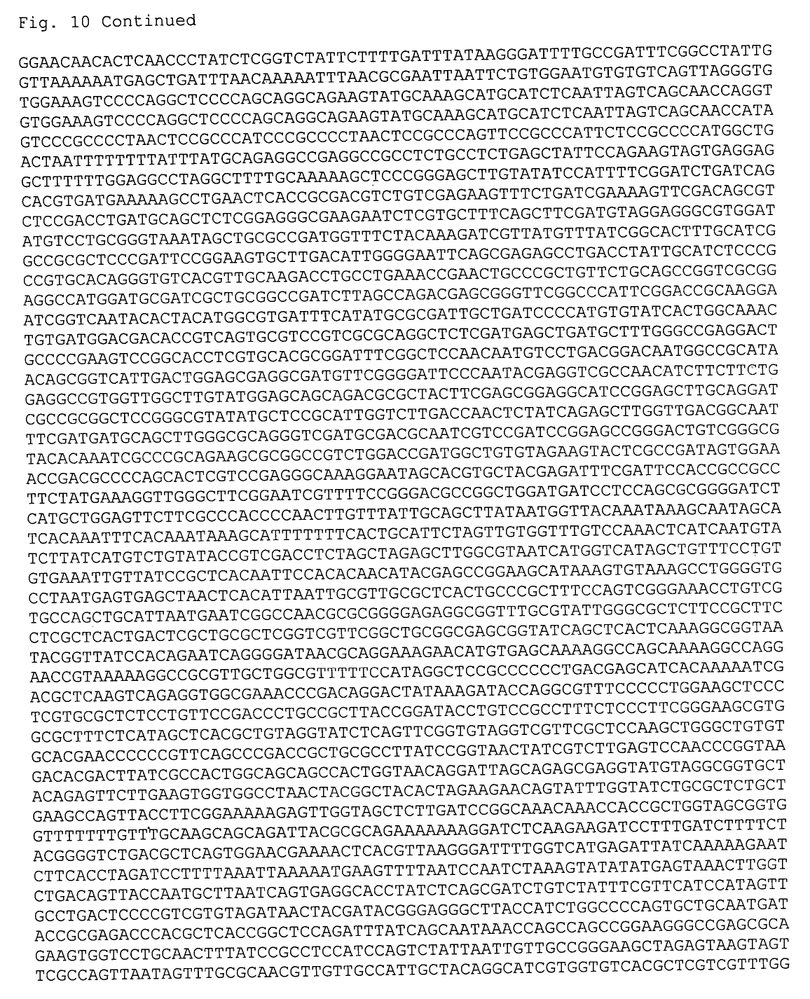

Next, the right arm homology region of pShuttle-Nhe-GAL is replaced with Ad sequences also containing the beginning of the E1 region in addition to the homology region. pShuttle-Nhe-GAL is digested with BglII and PmeI, filled in with DNA polymerase Klenow fragment and religated to produce pShuttle-Nhe-GAL-delE1. The latter plasmid lacks the right arm homology region. A fragment containing Ad 496-5780 is obtained by PCR amplification from plasmid pXC1 (Microbix Corporation, Toronto, ON). The nucleotide sequence of this plasmid is presented as FIG. 9. The latter PCR amplification is carried out using a forward primer (reading into the E1 gene) containing AscII and NotI restriction sites and a reverse primer containing a NotI restriction site. After digestion with NotI, the PCR fragment is ligated to NotI-digested pShuttle-Nhe-GAL-delE1 DNA to produce plasmid pShuttle-Nhe-GAL-E1. A clone is chosen in which the orientation of the inserted E1 sequence is the same as that of the truncated E1 sequence in the deleted right arm homology. In a subsequent subcloning step, a DNA fragment encompassing nucleotides 7677 to 323 of plasmid pGene/V5-His is PCR-amplified using appropriate primers also containing a AscII restriction site. After digestion of the PCR fragment with AscII, it is ligated to AscII-digested pShuttle-Nhe-GAL-E1 DNA. Recombinants are identified by restriction digestion and by nucleotide sequencing. Nucleotide sequencing permits the selection of a clone in which the inserted pGene/V5-His promoter sequence is correctly oriented relative to the E1 sequences. This clone is named pShuttle-Nhe-GAL-E1-GAL. Finally, the NotI site next to the inserted GAL4 site-containing promoter (inserted pGene/V5-His promoter sequence) is deleted by QuikChange mutagenesis. As a consequence, the resulting construct pShuttle-Nhe-GAL-E1-GAL-delNot contains a unique NotI site situated immediately downstream from the E1 sequences. To prepare recombinant Ad, pShuttle-Nhe-GAL-E1-GAL-delNot DNA is linearized by NotI digestion and is co-electroporated with pAdEasy-1 DNA (He et al. 1998. Proc. Natl. Acad. Sci. USA 95, 2509-2514) into E.coli BJ5183 cells. BJ5183 cells (Stratagene catalog no. 200154) have the cellular components necessary to carry out homologous recombination between the introduced viral sequences. Detailed methods for the generation of recombinant Ad plasmids and the subsequent production of recombinant Ad viruses are discussed in He et al. (Proc. Natl. Acad. Sci. USA 95, 2509-2514 (1998)) and in Stratagene manual“AdEasy™ Adenoviral Vector System” (revision no. 060002). Briefly, recombinant Ad plasmids are characterized by restriction digestion. After preparation of a sufficient amount of DNA of a correct recombinant, the DNA is digested with PacI to separate plasmid sequences and inserted sequences comprising the Ad sequences. To produce recombinant Ad, the digested DNA is transfected into 293E4pIX cells. 293E4pIX cells are 293 cells containing an MMTV promoter-driven E4 transcription unit. In the presence of dexamethasone, these cells provide both E1 and E4 functions that are needed to amplify the desired recombinant virus that has conditionally active E1A and E4 genes. Using standard technology (briefly described in He et al.; see also Graham, F. L., and Prevec, L. 1991. Manipulation of adenovirus vectors. In: Methods in Molecular Biology, Gene Transfer and Expression Protocols, vol.7, ed. E. J. Murray, The Humana Press Inc., Clifton, N.J., pp. 109-127), virus plaques are isolated, and viral stocks are prepared. Viral stocks can be purified by CsCl gradient centrifugation. Note that pAdEasy-1 contains all Ad sequences except for nucleotides 1-3,533 and 28,130-30,820. Hence, the recombinant virus described here contains a deletion of a nonessential E3 region. By using an Ad plasmid that contains all E3 sequences, a recombinant virus that has a complete E3 region could be obtained.

Recombinant Adenovirus 2 (rAd2)

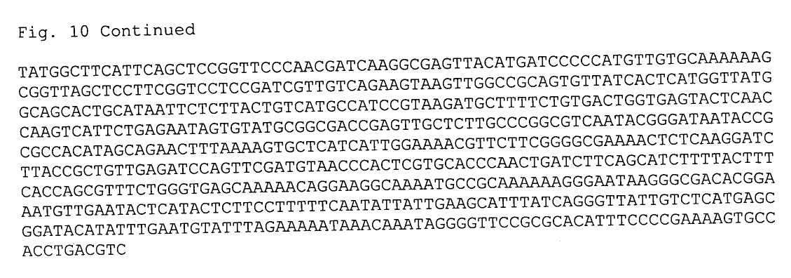

Recombinant adenovirus 2 is an E1/E3/E4-deleted Ad (lacking Ad 1-3,533, 28,130-30,820 and 35,586-35,808) that contains a transactivator that is related to transactivator GLVP described before. The transactivator employed in this example is a GAL4 DNA-binding domain-modified human progesterone receptor ligand-binding domain-human p65 activation domain chimera (GLP65) that can be retrieved from plasmid pSwitch (Invitrogen Corp.). The complete sequence of pSwitch is shown in FIG. 10. The transactivator is activated by estrogen antagonist mifepristone. In rAd2, the transactivator gene is functionally linked to an hsp70B-GAL4 tandem promoter. Optionally, rAd2 may also contain a passenger gene that is regulated by the hsp70B-GAL4 tandem promoter or another promoter.

hsp70B-GAL4 tandem promoter comprises a BamHI-HindIII fragment of about 460 bp length of the human hsp70B gene comprising the essential promoter sequences (about 350 bp), including the three functionally important heat shock element sequences, and an about 110 bp-long transcribed sequence (RNA leader region). Voellmy, R., et al. 1985. Proc. Natl. Acad. Sci. USA 82, 4949-4953. Schiller, P., et al. 1988. J. Mol. Biol. 203, 97-105. A unique restriction site was introduced close to the end of the transcribed sequence using the QuikChange procedure. Primers containing the same restriction site were used to PCR amplify the short intervening sequence (about 220 bp in length) present in plasmid phRL-CMV (Promega Corp., Madison, Wis.). After digestion, the PCR fragment was inserted into the added, unique restriction site in the hsp70B RNA leader region. An AscII site was then introduced into the intron sequence by the QuikChange procedure. A DNA fragment encompassing nucleotides 7677 to 323 of plasmid pGene/V5-His was PCR-amplified using appropriate primers also containing an AscII restriction site. After digestion of the PCR fragment with AscII, it was inserted into the intron AscII site. The resulting construct contained two promoters, an hsp70B promoter and a GAL4 site-containing promoter present in an intron that was inserted into the hsp70 RNA leader region. A gene placed downstream from this hsp70B-GAL4 tandem promoter can be transcribed from the hsp70B promoter that is activated by transient heat as well as from the GAL4 promoter that is activated by transactivator GLP65 in the presence of mifepristone. Note that a promoter that responds to heat and a GAL4 binding domain-containing transactivator does not need to be a tandem promoter. Hybrid promoters can also be constructed, e.g., by insertion of GAL4 binding sites into an hsp70B promoter. Note further that although a tandem or hybrid promoter provides the desired regulatory features that transcriptional activity of a linked gene is maintained subsequent to transient heat activation, in certain applications the use of such promoters is not absolutely required. In such applications, transactivator expression could be controlled, e.g., by the above-described 460-bp hsp70B promoter fragment.

To construct plasmid Shuttle-Nhe-hsp70B-GAL4-GLP65, a glp65-containing fragment encompassing nucleotides 500-2939 of pSwitch is PCR-amplified and inserted along with a fragment containing the Hsp70B-GAL4 tandem promoter into the multicloning site of pShuttle-Nhe. Restriction analysis and nucleotide sequencing are used to identify a clone, in which tandem promoter and glp65 sequences are joined in the proper relative orientation and in which transcription of glp65 proceeds clockwise, i.e., towards the right arm homology region. The pSwitch fragment contained in pShuttle-Nhe-hsp70B-GAL4-GLP65 includes the GLP65-coding sequence and a 3′ untranslated region from a bovine growth hormone gene.

The rAD2 construct can also include a passenger gene. In this example IL12-coding sequences and 3′ untranslated sequences are PCR-amplified from an appropriate construct. This PCR fragment and a fragment containing the Hsp70B-GAL4 tandem promoter is inserted in the multicloning site next to the transactivator cassette. Nucleotide sequence analysis is used to verify that the Hsp70-GAL4 promoter is properly situated to control transcription from the il12 gene. It is noted that a single tandem promoter may be used to control glp65 and il12 gene activity. In this case the sequence of elements inserted in pShuttle-Nhe could be as follows: Hsp70B/GAL4 tandem promoter—il12 gene—internal ribosome binding site—glp65 gene—3′ untranslated sequences. Attention should be paid to the positioning of a passenger gene that is regulated by a constitutively active promoter. In such a case, a passenger gene cassette should be inserted downstream from the glp65 gene or in an orientation that results in an opposite transcriptional direction from that of the glp65 gene. It is noted further that any passenger gene and associated 3′ untranslated sequences to be incorporated in rAd2 need to be screened for the presence of PmeI and PacI sites. If such sites are present, they need to be deleted. Deletion can typically be achieved conveniently by using the QuikChange mutagenesis procedure.

To prepare rAD2 (virus), rAD2 plasmid (pShuttle-Nhe-hsp70B-GAL4-GLP65 or a derivative plasmid) resulting from the above subcloning steps is linearized with PmeI, co-introduced with AdEasy-1 sequences in an appropriate E.coli strain to achieve homologous recombination of viral sequences, isolation of a correct recombinant plasmid, preparation of a sufficient amount of plasmid, digestion with PacI, transfection into 293 cells expressing E4, and isolation of rAd2 virus as described before.

Cells in a cell culture or cells in tissues infected with a mixture of rAd1 and rAd2 that are exposed to transient, directed heat in the presence of an appropriate concentration of mifepristone permit one round of replication of the virus pair (and expression of a passenger gene, if present, until cells are lysed). Viruses released from the initially infected cells infect surrounding cells. A second round of replication of the viruses can be triggered by a second transient, directed heat dose, and so forth.

Inevitably, in cells co-infected with rAd1 & 2, there is a low level of expression of GLP65 transactivator. This activity can be detected, e.g., by sensitive assays of passenger gene expression in the presence of mifepristone (without heat treatment). Any of the following measures reduces basal level expression of GLP65. Several of these measures can be combined for maximal effect. First, read-through transcriptional activity from tryptic, upstream promoters can be eliminated by inserting the tandem promoter—glp65 gene cassette in the opposite orientation, i.e., in the orientation in which the tandem promoter faces the right arm homology region of pShuttle-Nhe. Second, elimination of the 3′ untranslated sequences adjacent to the glp65 gene also reduces basal expression. This can be achieved by co-introducing into the multicloning site of pShuttle-Nhe an Hsp70-GAL4 tandem promoter and a shortened PCR fragment containing nucleotides 500-2494 of pSwitch. Third, the sequence around the AUG at which translation of GLP65 begins (position 519-521 in pSwitch) can be modified to reduce translation efficiency. A reduction is achieved by changing the A in position −3 (relative to the A of the AUG start codon) to a T, and the G at position +4 to a T.

All references cited in this application, including publications, patents and patent applications, shall be considered as having been incorporated in their entirety.

1. A modified, conditionally replication-competent virus whose genome includes a gene switch activatable in an infected cell by exposure of the cell to heat and a small molecule regulator, the gene switch controlling the expression of a gene for a viral protein required for efficient replication of the modified virus.

2. The modified virus of claim 1, wherein the viral genome further includes a passenger gene.

3. The modified virus of claim 1, wherein the virus is a member of a family selected from the group consisting of Adenoviridae, Herpesviridae, and Retroviridae.

4. The modified virus of claim 1, wherein the virus is an adenovirus.

5. The modified virus of claim 4, wherein the gene switch controls the expression of at least one viral protein selected from the group consisting of E1A, E1B and E4.

6. A modified, conditionally replication-competent virus whose genome includes a gene switch that is activated in an infected cell by exposure of the cell to heat and is repressed by exposure of the cell to a small molecule regulator, the gene switch controlling the expression of a gene for a viral protein required for efficient replication of the modified virus.

7. The modified virus of claim 6, wherein the viral genome further includes a passenger gene.

8. The modified virus of claim 6, wherein the virus is a member of a family selected from the group consisting of Adenoviridae, Herpesviridae, and Retroviridae.

9. The modified virus of claim 6, wherein the virus is an adenovirus.

10. The modified virus of claim 9, wherein the gene switch controls the expression of at least one viral protein selected from the group consisting of E1A, E1B and E4.

11. A pair of modified viruses whose combined genomes contain all genetic information required for conditional replication of the virus pair; including a gene switch activatable in an infected cell by exposure of the cell to heat and a small molecule regulator, the gene switch controlling the expression of a gene for a viral protein required for efficient replication of the virus pair.

12. The modified virus pair of claim 11, wherein the genome of at least one of the viruses further includes a passenger gene.

13. The modified virus pair of claim 11, wherein the viruses are members of a family selected from the group consisting of Adenoviridae, Herpesviridae, and Retroviridae.

14. The modified virus pair of claim 11, wherein both viruses are adenoviruses.

15. The modified virus pair according to claim 14, wherein the gene switch controls the expression of at least one viral protein selected from the group consisting of E1A, E1B and E4.

16. A pair of modified viruses whose combined genomes contain all genetic information required for conditional replication of the virus pair; including a gene switch that is activated in an infected cell by exposure of the cell to heat and is repressed by exposure of the cell to a small molecule regulator, the gene switch controlling the expression of a gene for a viral protein required for efficient replication of the virus pair.

17. The modified virus pair of claim 16, wherein the genome of at least one of the viruses further includes a passenger gene.

18. The modified virus pair of claim 16, wherein the viruses are members of a family selected from the group consisting of Adenoviridae, Herpesviridae, and Retroviridae.

19. The modified virus pair of claim 16, wherein both viruses are adenoviruses.

20. The modified virus pair according to claim 19, wherein the gene switch controls the expression of at least one viral protein selected from the group consisting of E1A, E1B and E4.


SEQUENCE LISTING (4 sequences)

SEQ ID NO: 1 - DNA, 6621 bp, Artificial
  pShuttle transfer vector
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatactggtaccgcggccgcctcgagtctagagatatcgaattcaagcttgtcgactcgaagatctgggcgtggttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagagggagtttaaacgaattcaatagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaacatgcatggatccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcagccatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattttgttaaaatttttgttaaatcagctcattttttaaccaataggccgaaatcggcaacatcccttataaatcaaaagaatagaccgcgatagggttgagtgttgttccagtttggaacaagagtccactattaaagaacgtggactccaacgtcaaagggcgaaaaaccgtctatcagggcgatggcccactacgtgaaccatcacccaaatcaagttttttgcggtcgaggtgccgtaaagctctaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgcgcgcttaatgcgccgctacagggcgcgtccattcgccattcaggatcgaattaattcttaattaa

SEQ ID NO: 2 - DNA, 7697 bp, Artificial
  pGene/V5-His plasmid
ccgagctcttacgcgggtcgaagcggagtactgtcctccgagtggagtactgtcctccgagcggagtactgtcctccgagtcgagggtcgaagcggagtactgtcctccgagtggagtactgtcctccgagcggagtactgtcctccgagtcgactctagagggtatataatggatctcgagatatcggagctcgtttagtgaaccgtcagatcgcctggagacgccatccacgctgttttgacctccatagaagacaccgggaccgatccagcctccgcggccgggaacggtgcattggaacgcgcattccccgtgttaattaacaggtaagtgtcttcctcctgtttccttcccctgctattctgctcaaccttcctatcagaaactgcagtatctgtatttttgctagcagtaatactaacggttctttttttctcttcacaggccaccaagcttggtaccgagctcggatccactagtccagtgtggtggaattctgcagatcgaaacgatgatagatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaagcggccgctcgaggtcacccattcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacatctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgcgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgta

SEQ ID NO: 3 - DNA, 9905 bp, Artificial
  pXC1 plasmid
cccttccagctctctgccccttttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcggcctgtcctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccgggcccccatttcccct

SEQ ID NO: 4 - DNA, 7323 bp, Artificial
  pSwitch plasmid
gacggatcgggagatcattcgagcttgcatgcctgcaggtcgaagcggagtactgtcctccgagtttaaaagcggagtactgtcctccgaggatatcagcggagtactgtcctccgagtcgcgaagcggagtactgtcctccgagatcgatgtcgaccccgcccagcgtcttgtcattggcgaattcgaacacgcagatgcagtcggggcggcgcggtccgaggtccacttcgcatattaaggtgacgcgtgtggcctcgaatcgcctggagacgccatccacgctgttttgacctccatagaagacaccgggaccgatccagcctccgcggccgggaacggtgcattggaacgcggattccccgtgttaattaacaggtaagtgtcttcctcctgtttccttcccctgctattctgctcaaccttcctatcagaaactgcagtatctgtatttttgctagcagtaatactaacggttctttttttctcttcacaggccaccaagctaccggtccaccatggactcccagcagccagatctgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttagaattcccgggtgtcgaccagaaaaagttcaataaagtcagagttgtgagagcactggatgctgttgctctcccacagccagtgggcgttccaaatgaaagccaagccctaagccagagattcactttttcaccaggtcaagacatacagttgattccaccactgatcaacctgttaatgagcattgaaccagatgtgatctatgcaggacatgacaacacaaaacctgacacctccagttctttgctgacaagtcttaatcaactaggcgagaggcaacttctttcagtagtcaagtggtctaaatcattgccaggttttcgaaacttacatattgatgaccagataactctcattcagtattcttggatgagcttaatggtgtttggtctaggatggagatcctacaaacacgtcagtgggcagatgctgtattttgcacctgatctaatactaaatgaacagcggatgaaagaatcatcattctattcattatgccttaccatgtggcagatcccacaggagtttgtcaagcttcaagttagccaagaagagttcctctgtatgaaagtattgttacttcttaatacaattcctttggaagggctacgaagtcaaacccagtttgaggagatgaggtcaagctacattagagagctcatcaaggcaattggtttgaggcaaaaaggagttgtgtcgagctcacagcgtttctatcaacttacaaaacttcttgataacttgcatgatcttgtcaaacaacttcatctgtactgcttgaatacatttatccagtcccgggcactgagtgttgaatttccagaaatgatgtctgaagttattgctgggtcgacgcccatggaattccagtacctgccagatacagacgatcgtcaccggattgaggagaaacgtaaaaggacatatgagaccttcaagagcatcatgaagaagagtcctttcagcggacccaccgacccccggcctccacctcgacgcattgctgtgccttcccgcagctcagcttctgtccccaagccagcaccccagccctatccctttacgtcatccctgagcaccatcaactatgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcctaaggatcctccggactagaaaagccgaattctgcaggaattgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggctcgagggggggcccgaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaagaatcttcacctagatccttttaaattaaaaatgaagttttaatccaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc